(19) United States
(12) Patent Application Publication
(10) Pub. No.: US 2025/0174325 A1
(43) Pub. Date: May 29, 2025

(54) METHODS AND SYSTEMS FOR DETECTING USER SKIN CONDITION AND GENERATING A PERSONALIZED REPORT

(71) Applicant: Samsung Electronics Co., Ltd., Suwon-si (KR)

(72) Inventor: Amardeep SINGH, Noida (IN)

(21) Appl. No.: 18/674,034

(22) Filed: May 24, 2024

Related U.S. Application Data
(63) Continuation of application No. PCT/KR2024/006997, filed on May 23, 2024.

(30) Foreign Application Priority Data
Nov. 7, 2023 (IN) 202311076074

(51) Int. Cl. G16H 15/00; G16H 40/67; G16H 50/30

(52) U.S. Cl. CPC G16H 15/00; G16H 40/67; G16H 50/30

(57) ABSTRACT

A method for generating a personalized report on skin condition of a user using a wearable device connected to a mobile device of the user is provided. The method includes monitoring skin attributes and environmental conditions associated with the user, determining, based on sensor-related data from the wearable device, a distance and an angle of the wearable device with reference to a position of the mobile device to identify hand movement of the user, determining parameters associated with the identified hand movement of the user, classifying the identified hand movement of the user as a scratching signature, determining scratching characteristics associated with the scratching signature of the user, determining a level of scratching and a deteriorating condition of the user's skin by comparing the scratching characteristics with predefined scratching characteristics and associated skin diseases information stored in a database, and generating the personalized report on the skin condition.

### CROSS-REFERENCE TO RELATED APPLICATION(S)

This application is a continuation application, claiming priority under § 365 (c), of an International application No. PCT/KR2024/006997, filed on May 23, 2024, which is based on and claims the benefit of an Indian Patent Application number 202311076074, filed on Nov. 7, 2023, in the Indian Patent Office, the disclosure of which is incorporated by reference herein in its entirety.

### FIELD OF THE INVENTION

The disclosure relates to methods and systems for predicting skin deteriorating condition of a user using a wearable device connected to a mobile device of the user.

### BACKGROUND

Wearable devices such as wearable smartwatches have become increasingly sophisticated with the integration of a diverse array of sensors. Such wearable devices, initially designed for tracking daily activity and providing smartphone notifications, have now found a significant role in the medical field. Equipped with sensors, the wearable smartwatches can monitor vital signs, detect irregularities, track physical activity, and even monitor user activities/actions. The expanding use of such wearable devices in the medical field offers healthcare professionals and patients valuable insights into health and well-being, enabling early detection of health issues, remote patient monitoring, and the potential to improve the overall quality of care through real-time data analysis and communication with healthcare providers. As the technology continues to evolve, wearable smartwatches hold great promise for revolutionizing healthcare by providing accessible and continuous health monitoring.

One of the major area in the domain of personal care and monitoring is skin related diseases that cause itching. Itching, or pruritus, is a common skin sensation that often results from various factors, including dry skin, insect bites, allergies, or underlying skin conditions. While mild itching is generally harmless, persistent, or severe itching can be a symptom of underlying skin diseases like eczema, psoriasis, or dermatitis. Early detection of these skin-related diseases associated with itching is crucial, as it can lead to timely treatment and management, preventing potential complications, and improving the patient's quality of life. Regular skin examinations, especially when itching persists, can help identify and address these conditions promptly, reducing the risk of long-term discomfort and complications.

Various solutions have been to track such itching. However, such solutions require complex hardware components such as various sensors, and/or imaging devices. Such solutions either require Infrared (IR) camera(s) that continuously monitor the user and/or require multiple patch-type sensors attached to the skin of the user. Thus, these solutions are cumbersome and not user-friendly.

Accordingly, there is a need to overcome at least the above challenges associated with conventional techniques of tracking itching and monitoring skin-related diseases.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide methods, systems, and computer-readable storage media for predicting skin deteriorating condition of a user using a wearable device connected to a mobile device of the user.

Additional aspects will be set forth in part in the description which follows and, in part, will be apparent from the description, or may be learned by practice of an embodiment of the disclosure.

In accordance with an aspect of the disclosure, a method for generating a personalized report on skin condition of a user using a wearable device connected to a mobile device of the user is provided. The method may include monitoring, using the wearable device, skin attributes, and environmental conditions associated with the user. The method may include determining, based on sensor-related data from at least one accelerometer sensor of the wearable device, a distance and an angle of the wearable device with reference to a position of the mobile device to identify hand movement of the user. The method may include determining, based on the sensor-related data from at least one accelerometer sensor of the wearable device, one or more parameters associated with the identified hand movement of the user. The method may include classifying the identified hand movement of the user as a scratching signature by correlating the determined one or more parameters with corresponding reference parameters associated with one or more predefined scratching patterns. The method may include determining, using the wearable device, one or more scratching characteristics associated with the scratching signature of the user. The method may include determining a level of scratching and a deteriorating condition of the user's skin by comparing the one or more scratching characteristics with one or more predefined scratching characteristics and associated skin diseases information stored in a database. The method may include generating the personalized report on the skin condition based on at least one of the determined level of scratching, the deteriorating condition of the user's skin, the skin attributes, and the environmental conditions associated with the user.

In accordance with an aspect of the disclosure, a system for predicting skin deteriorating condition of a user using a wearable device connected to a mobile device of the user is provided. The system may include memory storing one or more computer programs, and one or more processors communicably coupled with the memory, wherein the one or more computer programs include computer-executable instructions that, when executed by the one or more processors, cause the system to monitor, using the wearable device, skin attributes and environmental conditions associated with the user. The one or more processors cause the system to determine, based on sensor-related data from at least one accelerometer sensor of the wearable device, a distance and an angle of the wearable device with reference to a position of the mobile device to identify hand movement of the user. The one or more processors cause the system to determine, based on the sensor-related data from at least one accelerometer sensor of the wearable device, one or more parameters associated with the identified hand movement of the user. The one or more processors cause the system to classify the identified hand movement of the user as a scratching signature by correlating the determined one or more parameters with corresponding reference parameters associated with one or more predefined scratching patterns. The one or more processors cause the system to determine, using the wearable device, one or more scratching characteristics associated with the scratching signature of the user. The one or more processors cause the system to determine a level of scratching and a deteriorating condition of the user's skin by comparing the one or more scratching characteristics with one or more predefined scratching characteristics and associated skin diseases information stored in a database. The one or more processors cause the system to generate a personalized report on the skin condition based on at least one of the determined level of scratching, the deteriorating condition of the user's skin, the skin attributes, and the environmental conditions associated with the user.

In accordance with an aspect of the disclosure, one or more computer-readable storage media storing one or more computer programs including computer-executable instructions that, when executed by one or more processors of an electronic device for generating a personalized report on skin condition of a user using a wearable device connected to a mobile device of the user, cause the electronic device to perform operations are provided. The operations may include monitoring, using the wearable device, skin attributes, and environmental conditions associated with the user. The operations may include determining, based on sensor-related data from at least one accelerometer sensor of the wearable device, a distance and an angle of the wearable device with reference to a position of the mobile device to identify hand movement of the user. The operations may include determining, based on the sensor-related data from at least one accelerometer sensor of the wearable device, one or more parameters associated with the identified hand movement of the user. The operations may include classifying the identified hand movement of the user as a scratching signature by correlating the determined one or more parameters with corresponding reference parameters associated with one or more predefined scratching patterns. The operations may include determining, using the wearable device, one or more scratching characteristics associated with the scratching signature of the user. The operations may include determining a level of scratching and deteriorating condition of the user's skin by comparing the one or more scratching characteristics with one or more predefined scratching characteristics and associated skin diseases information stored in a database. The operations may include generating the personalized report on the skin condition based at least on the determined level of scratching, the deteriorating condition of the user's skin, the skin attributes, and the environmental conditions associated with the user.

In accordance with an aspect of the disclosure, a computer-readable storage medium storing instructions is provided. The instructions, when executed by at least one processor, may cause the at least one processor to perform the method corresponding.

Other aspects, advantages and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of an embodiment of the disclosure will be more apparent from the following description taken in conjunction with the accompanying drawings, in which:

FIG. 1 illustrates a schematic workflow of a system for generating a personalized report on skin condition of a user, according to an embodiment of the disclosure;

FIG. 2 illustrates a schematic block diagram of the system for generating a personalized report on the skin condition of a user, according to an embodiment of the disclosure;

FIG. 3 illustrates a flow chart of a method for generating the personalized report on the skin condition of the user, according to an embodiment of the disclosure;

FIG. 4 illustrates a graphical representation of different waveforms processed by a sensor processing unit of the system, according to an embodiment of the disclosure;

FIGS. 5A and 5B illustrate distance calculation by a distance calculator unit of the system, according to an embodiment of the disclosure;

FIGS. 6A and 6B illustrate angle calculation by a hand angle calculator unit of the system, according to an embodiment of the disclosure;

FIG. 7 illustrates an identification of potential scratching patterns by a user movement and hand posture identification unit of the system, according to an embodiment of the disclosure;

FIGS. 8A, 8B, 8C, 8D, 8E and 8F illustrate scenarios of identification of the hand movement and/or the hand posture, according to an embodiment of the disclosure;

FIG. 9 illustrates a process flow of operations of a scratching signature detector unit of the system, according to an embodiment of the disclosure;

FIGS. 10A, 10B, 10C, 10D, and 10E illustrate graphical representations corresponding to various mathematical features used by the scratching signature detector unit of the system, according to an embodiment of the disclosure;

FIG. 11 illustrates a process flow of operations of the signature analyzer unit of the system, according to an embodiment of the disclosure;

FIG. 12A illustrates a process flow of operations of the false scratching eliminator unit of the system, according to an embodiment of the disclosure;

FIG. 12B illustrates a graphical representation of waveforms representing scratching on a skin surface or a non-skin surface, according to an embodiment of the disclosure;

FIG. 13A illustrates a process flow of operations of a feature generator unit of the system, according to an embodiment of the disclosure;

FIG. 13B illustrates a graphical representation of waveforms representing a level of scratching and severity, according to an embodiment of the disclosure;

FIG. 14A illustrates a process flow of operations of a scratching evaluator unit of the system, according to an embodiment of the disclosure;

FIG. 14B illustrates a graphical representation of waveforms representing scratching on different body parts with associated frequencies, according to an embodiment of the disclosure;

FIG. 15 illustrates a schematic representation of a machine learning (ML) model utilizing a trained dataset to predict the presence of the skin disease in the user, according to an embodiment of the disclosure; and

FIG. 16 illustrates a flow chart of a method for generating a personalized report on the skin condition of a user, according to an embodiment of the disclosure.

The same reference numerals are used to represent the same elements throughout the drawings.

### DETAILED DESCRIPTION

The following description with reference to the accompanying drawings is provided to assist in a comprehensive understanding of an embodiment of the disclosure as defined by the claims and their equivalents. It includes various specific details to assist in that understanding, but these are to be regarded as merely exemplary. Accordingly, those of ordinary skill in the art will recognize that various changes and modifications of an embodiment described herein can be made without departing from the scope and spirit of the disclosure. In addition, descriptions of well-known functions and constructions may be omitted for clarity and conciseness.

The terms and words used in the following description and claims are not limited to the bibliographical meanings, but are merely used by the inventor to enable a clear and consistent understanding of the disclosure. Accordingly, it should be apparent to those skilled in the art that the following description of an embodiment of the disclosure is provided for illustration purposes only and not for the purpose of limiting the disclosure as defined by the appended claims and their equivalents.

It is to be understood that the singular forms “a”, “an”, and “the” include plural referents unless the context clearly dictates otherwise. Thus, for example, reference to “a component surface” includes reference to one or more of such surfaces.

It will be understood by those skilled in the art that the foregoing general description and the following detailed description are explanatory of the invention and are not intended to be restrictive thereof.

Reference throughout this specification to “an aspect”, “another aspect” or similar language means that a particular feature, structure, or characteristic described in connection with the embodiment is included in at least one embodiment of the disclosure. Thus, appearances of the phrase “in an embodiment”, “in another embodiment” and similar language throughout this specification may, but do not necessarily, all refer to the same embodiment.

The terms “comprise”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components preceded by “comprises . . . a” does not, without more constraints, preclude the existence of other devices or other sub-systems or other elements or other structures or other components or additional devices or additional sub-systems or additional elements or additional structures or additional components.

As used here, terms and phrases such as “have”, “may have”, “include”, or “may include” a feature (like a number, function, operation, or component such as a part) indicate the existence of the feature and do not exclude the existence of other features. Also, as used here, the phrases “A or B”, “at least one of A and/or B”, or “one or more of A and/or B” may include all possible combinations of A and B. For example, “A or B”, “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of the disclosure.

An embodiment of the disclosure is directed towards a method and a system for generating a personalized report on skin condition of a user using a wearable device connected to a mobile device of the user. A key objective of the disclosure is to utilize on-device Artificial Intelligence (AI) model for tracking a scratching pattern of the user and predicting skin deteriorating condition. The method comprises utilizing sensors embedded in the wearable devices and the mobile device to capture accelerometer data, for measuring the acceleration of the wearable device along three axes. This accelerometer data is classified to identify potential scratching patterns from hand movements using various parameters such as jerks, motion, frequency, etc. Further, the potential scratching pattern is evaluated to identify scratching characteristics (such as, frequency, severity, increasing/decreasing) by checking if a current waveform pattern within a time interval is similar to the past waveform pattern. Furthermore, the user is provided with a personalized report having information related to a skin deteriorating level and the probability of the skin disease determined based on the identified scratching characteristics.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an integrated circuit (IC), or the like.

The processor may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

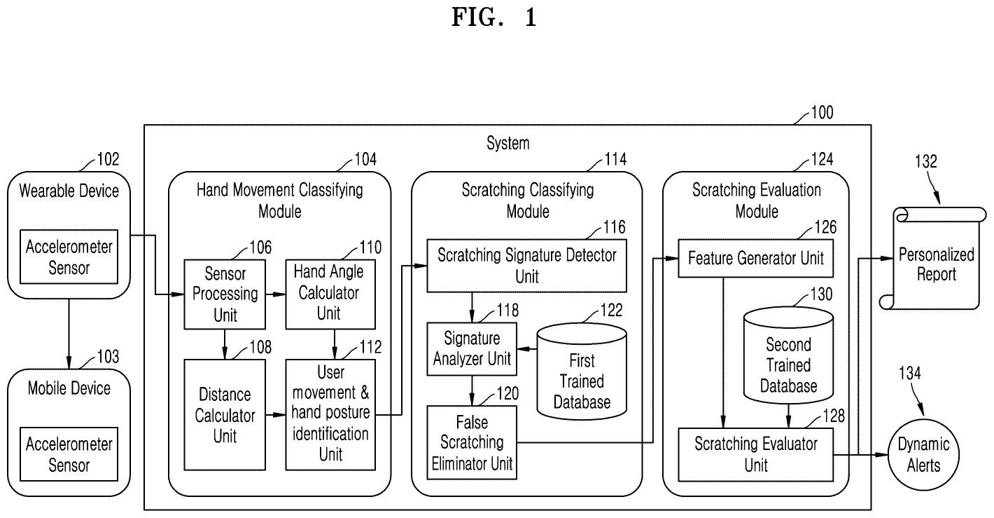

FIG. 1 illustrates a schematic workflow of a system for generating a personalized report on skin condition of a user, according to an embodiment of the disclosure.

Referring to FIG. 1, the system 100 may be a standalone entity which is communicably coupled to a wearable device 102 and/or a mobile device 103 via a network. According to an embodiment of the disclosure, the system 100 either in-part or as a whole may be implemented at the wearable device 102 and/or the mobile device 103. Examples of the wearable device 102 may include, but are not limited to, a smartwatch, a smart ring, a smart band, or the like. Examples of the mobile device 103 may include, but are not limited to, a smartphone, a tablet, a portable computing device, or any other portable computing device embedded with at least an accelerometer sensor.

The wearable device 102 may include at least one accelerometer sensor configured to generate accelerometer data related to an acceleration of the wearable device 102 along three axes (e.g., x, y, and z). The wearable device 102 may also include other essential standard components, such as a display, a speaker, a processor, memory, and one or more other required sensors (such as, heart rate monitors, temperature sensors, and so forth). However, a detailed description of such components has been omitted for the same of the brevity.

The mobile device 103 may include at least one accelerometer sensor configured to generate accelerometer data related to an acceleration of the mobile device 103 along three axes (e.g., x, y, and z). A detailed description of the architecture and standard operations on the mobile device 103 has been omitted, just for the sake of brevity.

The system 100 may be communicably coupled with the wearable device 102 and/or the mobile device 103 and may be configured to receive the accelerometer data corresponding to each of the wearable device 102 and the mobile device 103. The system 100 may be configured to utilize a smart learning model that keeps track of the user's skin attributes and information related to current environment conditions of the user to generate the personalized report 132 and/or dynamic alerts 134. The system 100 may include a hand movement classifying module 104. The hand movement classifying module 104 may be configured to calculate a distance and/or an angle of the wearable device with respect to the mobile device 103 using the received accelerometer data to determine a hand movement and a posture of the hand of the user. The hand movement classifying module 104 may include a sensor processing unit 106, a hand angle calculator unit 110, a distance calculator unit 108, and a user movement and hand posture identification unit 112. The sensor processing unit 106 may be configured to take the received accelerometer data as input and remove noise and minor shaking movement. The sensor processing unit 106 may pre-process the accelerometer data prior to being utilized by the other unit(s) and/or module(s) of the system 100.

The distance calculator unit 108 may be configured to receive the pre-processed accelerometer data from the sensor processing unit 106 to calculate a distance between the mobile device 103 and the wearable device 102. The distance calculator unit 108 may determine the position of the mobile device 103 in a 3-Dimensional (3D) space based on the received accelerometer data corresponding to the mobile device 103 and map the position of the mobile device 103 as a reference point. The distance calculator unit 108 then determines the distance of the wearable device 102 from the reference point.

The hand angle calculator unit 110 may be configured to determine an angle of the user's hand with respect to the mobile device 103 from a reference angle point based on the received accelerometer data and acceleration determined due to a change in gravity. By utilizing the received accelerometer data and the acceleration due to the change in gravity, the hand angle calculator unit 110 may eliminate false cases.

The user movement and hand posture identification unit 112 may be communicably coupled with the sensor processing unit 106, the distance calculator unit 108, and/or the hand angle calculator unit 110, and configured to classify the user's movement and hand gestures as with or within a body range. The user movement and hand posture identification unit 112 may be configured to correlate the determined distance and the angle with corresponding predefined thresholds to identify the hand movement and/or hand posture of the user.

The system 100 may include a scratching classifying module 114 to classify the identified hand movement and/or hand posture of the user as a scratching signature or a non-scratching signature. The scratching signature may include, but is not limited to, neck scratching, head scratching, face scratching, arm scratching, thigh scratching, leg scratching, and random scratching. The non-scratching signature may include, but is not limited to, random tapping on a table, typing, and/or other random movements of the user's hand. The scratching classifying module 114 may include a scratching signature detector unit 116, a signature analyzer unit 118, a false scratching eliminator unit 120, and a first trained database 122.

The scratching signature detector unit 116 may be configured to identify scratching signatures from the identified and/or classified hand movements and/or hand posture of the user based on one or more features/parameters and pre-stored patterns. The one or more features/parameters may include, but are not limited to, frequency, amplitude, motion, jerks, and the like. The scratching signature detector unit 116 may identify the one or more predefined features based on the received accelerometer data corresponding to the wearable device 102 and/or the mobile device 103.

The signature analyzer unit 118 may be configured to co-relate the one or more parameters with corresponding mathematical features stored in the first trained database 122. The mathematical features may include, but are not limited to, an Inter Quartile Range (IQR), a Peak to Peak Range (PTP), an entropy, a Kurtosis, a Singular Value Decomposition (SVD) entropy, a line integral, a skewness, a mean, a standard deviation, a Root Mean Square (RMS), etc. The first trained database 122 may include various pre-stored scratching patterns and associated parameters/mathematical features. The first trained database 122 may also include differentiation of various false scratching patterns based on associated skin attributes and environmental conditions. According to an embodiment of the disclosure, the wearable device 102 and/or mobile device 103 may determine environmental conditions associated with the user. The signature analyzer unit 118 may utilize said determined environmental conditions and pre-stored environmental conditions in the trained database to classify the scratching patterns.

The false scratching eliminator unit 120 may be configured to remove all the false cases of the identified scratching patterns based on the one or more parameters and the information stored in the first trained database 122. The false scratching eliminator unit 120 may be configured to identify and remove non-skin scratching patterns. The signature analyzer unit 118 and/or the false scratching eliminator unit 120 may implement one or more ML models to train and utilize the first trained database 122.

The system 100 may include a scratching evaluation module 124. The scratching evaluation module 124 may be configured to evaluate the classified scratching signatures to identify one or more corresponding scratching characteristics such as, but not limited to, frequency, severity, increasing pattern/decreasing pattern, and the like. The scratching evaluation module 124 may compare a current waveform of each of the classified scratching signatures within a predefined time interval with a corresponding previous waveform pattern to identify the one or more corresponding scratching characteristics.

The scratching evaluation module 124 may include a feature generator unit 126 configured to generate one or more features based on refined data corresponding to the classified scratching signatures to predict the skin deterioration condition of the user.

The scratching evaluation module 124 may include a scratching evaluator unit 128 configured to compare the generated one or more features of data with a trained dataset stored in a second trained database 130 to generate the final personalized report 132 and/or dynamic alert(s) 134 when the skin condition of the user deteriorate. The second trained database 130 may include information corresponding to various scratching patterns associated with various skin diseases and corresponding conditions. The scratching evaluator unit 128 may be configured to determine a level of scratching and deteriorating condition of the user's skin by comparing the one or more scratching characteristics with skin disease information stored in the first trained database 122 using one or more ML models.

The personalized report 132 may include information related to skin deteriorating levels and the probability of the skin disease presence in the user. The dynamic alerts 134 may correspond to audio/video alerts based on the personalized report 132. The personalized report 132 may be shared with the user and/or a concerned medical practitioner to assist the user. The dynamic alerts 134 may be generated on the wearable device 102 and/or the mobile device 103. Some non-limiting examples of the dynamic alerts 134 are “scratching detecting on arms”, “Alert! skin started deteriorating”, and so forth.

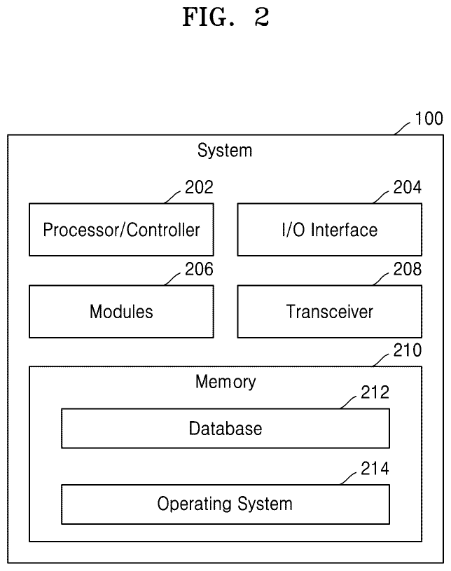

FIG. 2 illustrates a schematic block diagram of a system for generating a personalized report on the skin condition of a user, according to an embodiment of the disclosure.

Referring to FIG. 2, the system 100 may include a processor/controller 202, an Input/Output (I/O) interface 204, one or more modules 206, a transceiver 208, and memory 210. In an embodiment, the processor/controller 202 may be operatively coupled to each of the I/O interface 204, the modules 206, the transceiver 208, and the memory 210.

The processor/controller 202 may include at least one data processor for executing processes in the Virtual Storage Area Network. The processor/controller 202 may include specialized processing units such as integrated system (bus) controllers, memory management control units, floating point units, graphics processing units, digital signal processing units, etc. The processor/controller 202 may include a Central Processing Unit (CPU), a Graphics Processing Unit (GPU), or both. The processor/controller 202 may be one or more general processors, Digital Signal Processors (DSPs), Application-Specific Integrated Circuits (ASIC), Field-Programmable Gate Arrays (FPGAs), servers, networks, digital circuits, analog circuits, combinations thereof, or other now known or later developed devices for analyzing and processing data. The processor/controller 202 may execute a software program, such as code generated manually (e.g., programmed) to perform the desired operation.

The processor/controller 202 may be disposed in communication with one or more I/O devices via the I/O interface 204. The I/O interface 204 may employ communication Code-Division Multiple Access (CDMA), High-Speed Packet Access (HSPA+), Global System for Mobile communications (GSM), Long-Term Evolution (LTE), Worldwide interoperability for Microwave Access (WiMAX), or the like, etc.

Using the I/O interface 204, the system 100 may communicate with one or more I/O devices, specifically, to the wearable device 102 and/or the mobile device 103. Other examples of the input device may be an antenna, microphone, touch screen, touchpad, storage device, transceiver, video device/source, etc. The output devices may be a printer, fax machine, video display (e.g., Cathode Ray Tube (CRT), Liquid Crystal Display (LCD), Light-Emitting Diode (LED), plasma, Plasma Display Panel (PDP), Organic Light-Emitting Diode display (OLED) or the like), audio speaker, etc.

The processor/controller 202 may be disposed in communication with a communication network via a network interface. The network interface may be the I/O interface 204. The network interface may connect to the communication network to enable connection of the system 100 with the outside environment and/or device/system. The network interface may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. The communication network may include, without limitation, a direct interconnection, Local Area Network (LAN), Wide Area Network (WAN), wireless network (e.g., using Wireless Application Protocol), the internet, etc. Using the network interface and the communication network, the system 100 may communicate with other devices.

The processor/controller 202 may be configured to monitor the one or more skin attributes and the environmental conditions associated with the user using the wearable device 102 and/or the mobile device 103. Examples of the one or more skin attributes may include, but are not limited to, softness, moisture level, and a dryness level of the user's skin. Non-limiting examples of the environmental conditions may include a humidity level and a temperature associated with the surroundings of the user. The processor/controller 202 may be configured to determine a distance, and an angle of the wearable device 102 with reference to the position of the mobile device 103 to identify hand movement of the user based on sensor-related data from at least one accelerometer sensor of the wearable device 102 and the mobile device 103. The processor/controller 202 may be configured to determine one or more parameters associated with the identified hand movement of the user based on the sensor-related data. The one or more parameters associated with the identified hand movement of the user may include, but are not limited to, frequency, amplitude, motion, and jerks.

The processor/controller 202 may be configured to classify the identified hand movement of the user as a scratching signature by correlating the determined one or more parameters with corresponding reference parameters associated with one or more predefined scratching patterns. To classify the identified hand movement of the user as the scratching signature, the processor/controller 202 may be configured to compare the determined distance and the angle of the wearable device with corresponding predefined thresholds. Upon determining that the determined distance and the angle of the wearable device is less than the corresponding predefined thresholds, the processor/controller 202 may classify the identified hand movement of the user as the scratching signature. However, upon determining that at least one of the determined distance and the angle of the wearable device is greater than the corresponding predefined thresholds, the processor/controller 202 may classify the identified hand movement of the user as a non-scratching signature. Examples of the scratching signature may include, but are not limited to, neck scratching, head scratching, face scratching, arm scratching, thigh scratching, leg scratching, and random scratching.

The processor/controller 202 may be configured to determine one or more scratching characteristics associated with the scratching signature of the user using the wearable device 102. Some non-limiting examples of the one or more scratching characteristics may include an area of scratching, a frequency of scratching, and an intensity of scratching. The processor/controller 202 may utilize one or more sensors (such as, accelerometer, gyroscope, etc.) of the wearable device 102 to determine the one or more scratching characteristics associated with the scratching signature of the user.

The processor/controller 202 may be configured to determine a level of scratching and deteriorating condition of the user's skin by comparing the one or more scratching characteristics with one or more predefined scratching characteristics and associated skin disease information stored in a database (e.g., the second trained database 130). The processor/controller 202 may be configured to generate the personalized report 132 on the skin condition based on the determined level of scratching, the deteriorating condition of the user's skin, the skin attributes, and the environmental conditions associated with the user. To determine the deteriorating condition of the user's skin, the processor/controller 202 may be configured to monitor the identified hand movement for a predefined time duration. The processor/controller 202 may be configured to identify one or more changes in the identified hand movement during the predefined time duration. The processor/controller 202 may correlate the identified one or more changes with the environmental conditions to remove any false cases and determine the deteriorating condition of the user's skin. The processor/controller 202 may be configured to the dynamic alert 134 (also referred to as “the alert 134”) based on the generated personalized report 132.

The processor/controller 202 may implement various techniques such as, but not limited to, data extraction, Artificial Intelligence (AI), Machine Learning (ML), Deep Learning (DL), and so forth, to achieve the desired objective.

The memory 210 may be communicatively coupled to the at least one processor/controller 202. The memory 210 may be configured to store data, and instructions executable by the at least one processor/controller 202. The memory 210 may communicate via a bus within the system 100. The memory 210 may include, but not limited to, a computer-readable storage media (e.g. a non-transitory computer-readable storage media), such as various types of volatile and non-volatile storage media including, but not limited to, Random Access Memory (RAM), Read-Only Memory (ROM), Programmable Read-Only Memory (PROM), electrically Programmable ROM, electrically erasable ROM, flash memory, magnetic tape or disk, optical media, and the like. For example, the memory 210 may include a cache or random-access memory for the processor/controller 202. For example, the memory 210 may be separate from the processor/controller 202, such as a cache memory of a processor, the system memory, or other memory. The memory 210 may be an external storage device or database for storing data. The memory 210 may be operable to store instructions executable by the processor/controller 202. The functions, acts, or tasks illustrated in the figures or described may be performed by the programmed processor/controller 202 for executing the instructions stored in the memory 210. The functions, acts or tasks are independent of the particular type of instruction set, storage media, processor or processing strategy and may be performed by software, hardware, integrated circuits, firmware, micro-code, and the like, operating alone or in combination. Likewise, processing strategies may include multiprocessing, multitasking, parallel processing, and the like.

According to an embodiment of the disclosure, the modules 206 may be included within the memory 210. The memory 210 may include a database 212 to store data. The database 212 may correspond to the first trained database 122 and/or the second trained database 130. The one or more modules 206 may include a set of instructions that may be executed to cause the system 100 to perform any one or more of the methods/processes disclosed herein. In an embodiment, the modules 104, 114, and 124 (as shown in FIG. 1) may be a part of the modules 206. The modules 206 may be configured to perform the steps of the disclosure using the data stored in the database 212, for performing the desired objective of the disclosure as discussed herein. According to an embodiment of the disclosure, each of the modules 206 may be a hardware unit that may be outside the memory 210. The memory 210 may include an operating system 214 for performing one or more tasks of the system 100, as performed by a generic operating system in the communications domain. The transceiver 208 may be configured to receive and/or transmit signals to and from the wearable device 102 associated with the user. In one embodiment, the database 212 may be configured to store the information as required by the one or more modules 206 and the processor/controller 202 to perform one or more desired functions.

The disclosure contemplates a computer-readable medium that includes instructions or receives and executes instructions responsive to a propagated signal. The instructions may be transmitted or received over the network via a communication port or interface or using a bus. The communication port or interface may be a part of the processor/controller 202 or may be a separate component. The communication port may be created in software or may be a physical connection in hardware. The communication port may be configured to connect with a network, external media, the display, or any other components in the system, or combinations thereof. The connection with the network may be a physical connection, such as a wired Ethernet connection, or may be established wirelessly. Likewise, the additional connections with other components of the system 100 may be physical or may be established wirelessly. The network may alternatively be directly connected to the bus. For the sake of brevity, the architecture, and standard operations of the operating system 214, the memory 210, the database 212, the processor/controller 202, the transceiver 208, and the I/O interface 204 are not discussed in detail.

The one or a plurality of processors control the processing of the input data in accordance with a predefined operating rule or artificial intelligence (AI) model stored in the non-volatile memory and the volatile memory. The predefined operating rule or artificial intelligence model is provided through training or learning.

Here, being provided through learning means that, by applying a learning technique to a plurality of learning data, a predefined operating rule or AI model of the desired characteristic is made. The learning may be performed in a device itself in which AI according to an embodiment is performed, and/or may be implemented through a separate server/system.

The AI model may consist of a plurality of neural network layers. Each layer has a plurality of weight values and performs a layer operation through the calculation of a previous layer and an operation of a plurality of weights. Examples of neural networks include, but are not limited to, Convolutional Neural Network (CNN), Deep Neural Network (DNN), Recurrent Neural Network (RNN), Restricted Boltzmann Machine (RBM), Deep Belief Network (DBN), Bidirectional Recurrent Deep Neural Network (BRDNN), Generative Adversarial Networks (GAN), and deep Q-networks.

The learning technique is a method for training a predetermined target device (for example, a robot) using a plurality of learning data to cause, allow, or control the target device to make a determination or prediction. Examples of learning techniques include, but are not limited to, supervised learning, unsupervised learning, semi-supervised learning, or reinforcement learning.

According to the disclosure, in a method for predicting the skin deteriorating condition of the user, the method may include using an artificial intelligence model to recommend/execute the plurality of instructions. The processor may perform a pre-processing operation on the data to convert the data into a form appropriate for use as an input for the artificial intelligence model. The artificial intelligence model may be obtained by training. Here, “obtained by training” means that a predefined operation rule or artificial intelligence model configured to perform the desired feature (or purpose) is obtained by training a basic artificial intelligence model with multiple pieces of training data by a training technique. The artificial intelligence model may include a plurality of neural network layers. Each of the plurality of neural network layers may include a plurality of weight values and performs neural network computation by computation between a result of computation by a previous layer and the plurality of weight values.

Reasoning prediction is a technique of logical reasoning and predicting by determining information and includes, e.g., knowledge-based reasoning, optimization prediction, preference-based planning, or recommendation.

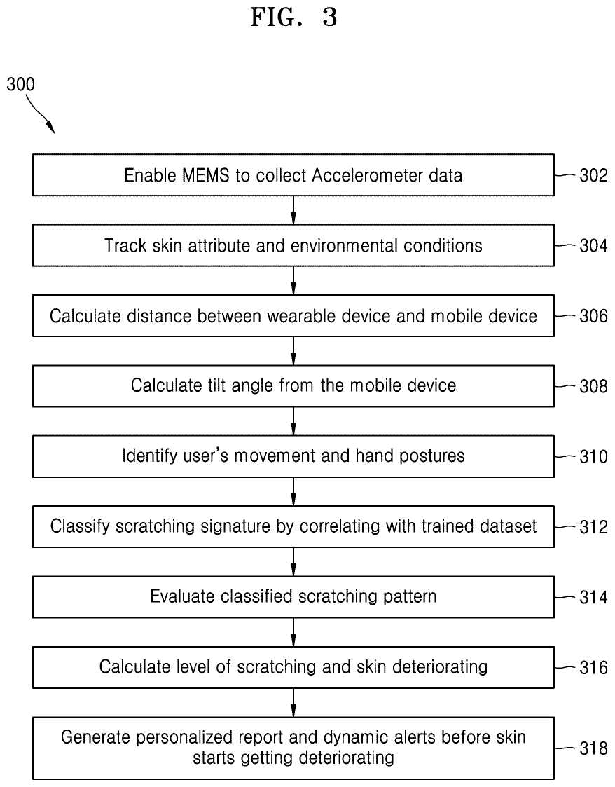

FIG. 3 illustrates a flow chart of a method for generating the personalized report on the skin condition of the user, according to an embodiment of the disclosure.

Referring to FIG. 3, the method 300 may be implemented by one or more components of the system 100. However, for the sake of brevity, the method steps of the method 300 have been explained as being implemented by the system 100.

At operation 302, the system 100 may enable Micro-Electro-Mechanical Systems (MEMS) to collect accelerometer data using the wearable device 102 and/or the mobile device 103. The MEMS may correspond to MEMS accelerometers configured to collect the acceleration data. The accelerometer data may include a magnitude and/or a direction of the acceleration. The MEMS may correspond to a three-axes accelerometer sensor that may be triggered at a sampling rate of 10 ms to provide 100 readings in 1 second for analysis.

At operation 304, the system 100 may track the one or more skin attributes and the environmental conditions. The system 100 may track/monitor the skin attributes (e.g., dryness, moisture level etc.) along with environmental parameters (e.g., humidity, temperature, etc.) to rectify false positive itching/scratching pattern. Specifically, the system 100 may track the skin attributes and the environmental conditions to eliminate the scratching patterns that are common and obvious and may not cause any serious harm to the user.

At operation 306, the system 100 may calculate the distance between the wearable device 102 and the mobile device 103. The system 100 may calculate the distance between the wearable device 102 and the mobile device 103 based on the corresponding accelerometer data. The system 100 may utilize the calculated distance to determine whether the user's hand movement is within a predefined range that may indicate that the user's hand movement is within the body range of the user. For instance, if the distance is within the predefined threshold the system 100 may identify that the hand movement could be a case of scratching a body part.

At operation 308, the system 100 may calculate a tilt angle of the wearable device 102 with respect to the mobile device 103. In one embodiment, the tilt angle of the wearable device 102 may correspond to an angle of the wearable device 102 with respect to the force of gravity.

At operation 310, the system 100 may identify the user's movement and hand postures. In an embodiment, the system 100 may identify the user's hand movement and posture and the associated features such as, motion, frequency, amplitude, jerks, and other features based on the calculated distance and the tilt angle.

At operation 312, the system 100 may classify the user's hand movement and posture as a scratching signature by correlating with a trained dataset stored in the first trained database 122. Specifically, the system 100 may eliminate possible false cases (such as scratching on a table or typing, etc.) and classify the user's hand movement and posture as a scratching signature using the first trained database 122.

At operation 314, the system 100 may evaluate the classified scratching pattern. The system 100 may evaluate the classified scratching pattern based on a trained dataset stored in the second trained database 130 to enable prediction of any skin related diseases for which the user is scratching frequently without being aware of deterioration conditions of the skin.

At operation 316, the system 100 may calculate a level of scratching and skin deteriorating condition. The system 100 may determine the level of scratching based on scratching characteristics (e.g., frequency, intensity, etc.) over a period of time to predict the deteriorating condition of the skin of the user.

At operation 318, the system 100 may generate the personalized report 132 and the dynamic alerts 134 to alert the user before the user's skin starts deteriorating. In an embodiment, the system 100 may generate the dynamic alerts 134 based on the scratching signatures and corresponding frequency and intensity. The system 100 may generate the dynamic alerts 134 before the skin starts deteriorating.

Thus, the system 100 may be able to prevent the user from a serious skin related disease that may be caused due to careless scratching of the skin by the user. Further, the system 100 may timely make the user aware about the presence of a skin-related disease that causes itching/scratching sensation among the user. Thus, the system 100 may assist the user in taking timely action/treatment for the skin-related disease that the user may be unable to.

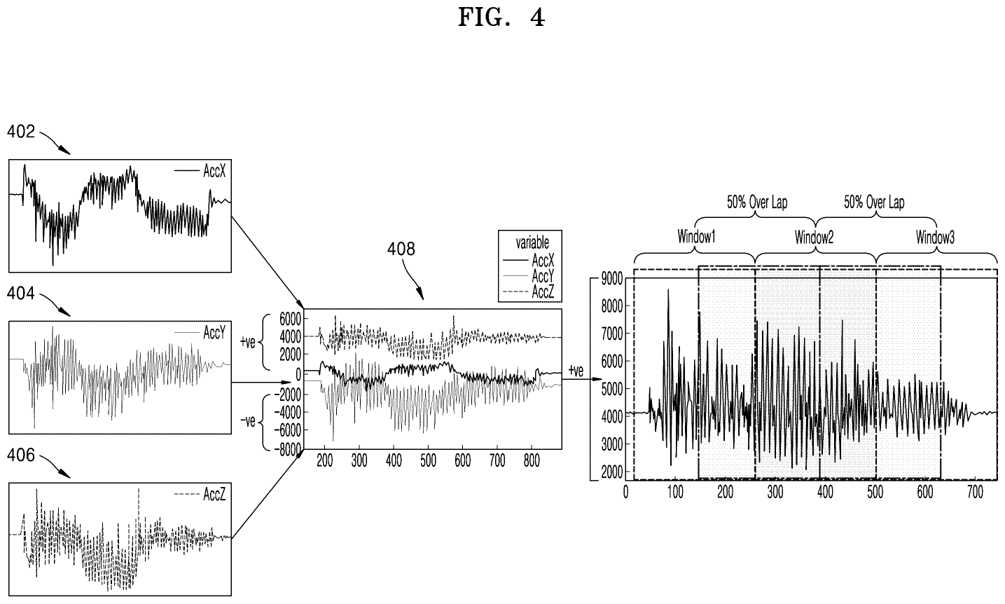

FIG. 4 illustrates a graphical representation of different waveforms processed by the sensor processing unit 106 of the system 100, according to an embodiment of the disclosure.

Referring to FIG. 4, waveforms 402-406 may correspond to the accelerometer data as received from the accelerometer sensor of the wearable device 102. The waveform 402 may correspond to the acceleration of the wearable device 102 in an x-direction. The waveform 404 may correspond to the acceleration of the wearable device 102 in a y-direction. The waveform 406 may correspond to the acceleration of the wearable device 102 in a z-direction. A block 408 may represent a combination of the waveforms 402-406 that may correspond to the received accelerometer data. In a non-limiting embodiment, the received accelerometer data may be represented as below Table 1:

TABLE 1

| TimeStamp | AccX | AccY | AccZ |
|---|---|---|---|
| 1680404342277 | 386 | −698 | 4065 |
| 1680404342317 | 401 | −684 | 4034 |
| 1680404342358 | 354 | −704 | 4036 |
| 1680404342397 | 394 | −702 | 4033 |
| 1680404342438 | 371 | −718 | 4015 |

The sensor processing unit 106 may receive 1133 rows of above-rows for the acceleration data over a 20 s time period at a sampling rate of 40 ms. The sensor processing unit 106 may consider both a positive (+ve) and a negative (−ve) direction of all three axes (x, y, z). The sensor processing unit 106 may calculate a square root of the sum of the squares of all three axes to obtain a magnitude vector that may represent the length of the waveforms to take into account to eliminate noise and random shaking from the acceleration data. In an embodiment, the magnitude vector may be represented using the following Equation 1:

$$\sqrt{{\sum }_{i = X}^{Z} {Acc ( i )}^{2}} = \sqrt{{AccX}^{2} + {AccY}^{2} + {AccZ}^{2}} \qquad Equation 1$$

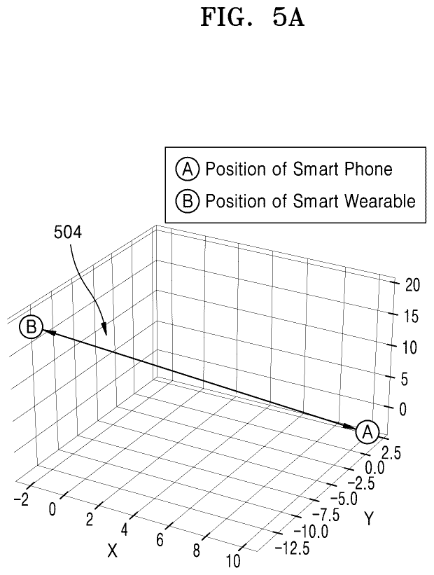

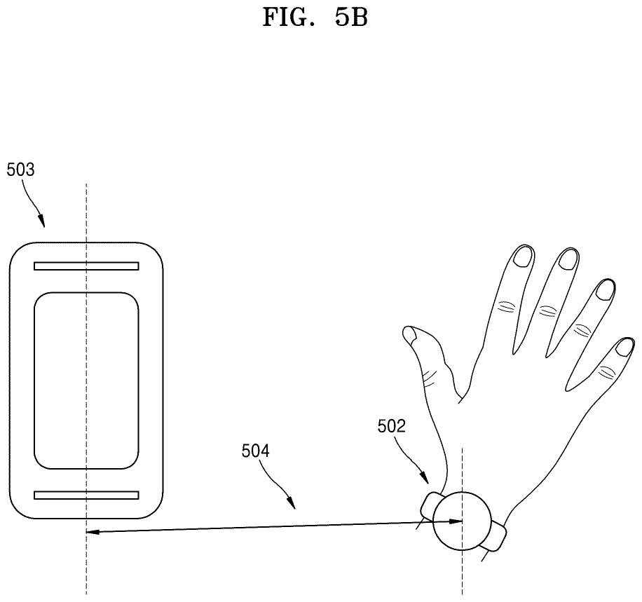

FIGS. 5A and 5B illustrate distance calculation by the distance calculator unit 108 of the system 100, according to an embodiment of the disclosure.

Referring to FIGS. 5A and 5B, the distance calculator unit 108 may be configured to identify a distance 504 between a wearable device 502 and a mobile device 503. The wearable device 502 and the mobile device 503 may correspond to the wearable device 102 and the mobile device 103, respectively.

The distance calculator unit 108 may use the acceleration data to determine the distance 504. The distance calculator unit 108 may perform an integration operation on the acceleration data to determine a velocity corresponding to each of the wearable device 502 and the mobile device 503. The distance calculator unit 108 may perform an integration operation of the calculated velocity data to determine the position of the wearable device 502 and the mobile device 503 in the 3-D space. Equations Equation 2-Equation 9 used by the distance calculator unit 108 are as follows:

$$Acc ( x , y , z ) = \frac{d [ Velocity ( X , Y , Z ) ]}{dt} \qquad Equation 2$$

$$d [ Velocity ( X , Y , Z ) ] = Acc ( x , y , z ) dt \qquad Equation 3$$

$${\int }_{t}^{t + 0.04} d [ Velocity ( X , Y , Z ) ] = {\int }_{t}^{t + 0.04} Acc ( x , y , z ) dt \qquad Equation 4$$

$$Velocity ( X , Y , Z ) = ( Acc ( x , y , z ) [ t + 0.04 ] - Acc ( x , y , z ) [ t ] ) dt \qquad Equation 5$$

$$Velocity ( X , Y , Z ) = ( d [ Position ( X , Y , Z ) ] ) / dt \qquad Equation 6$$

$$d [ Position ( X , Y , Z ) ] = Velocity ( X , Y , Z ) dt \qquad Equation 7$$

$${\int }_{t}^{t + 0.04} d [ Position ( X , Y , Z ) ] = {\int }_{t}^{t + 0.04} Velocity ( X , Y , Z ) dt \qquad Equation 8$$

$$Position ( X , Y , Z ) = Velocity ( X , Y , Z ) [ t + 0.04 ] - Velocity ( X , Y , Z ) [ t ] ) dt \qquad Equation 9$$

These are exemplary in nature and the distance calculator unit 108 may use any suitable technique and/or equation(s) to determine the positions of the wearable device 502 and the mobile device 503, and/or the distance 504 between the wearable device 502 and the mobile device 503.

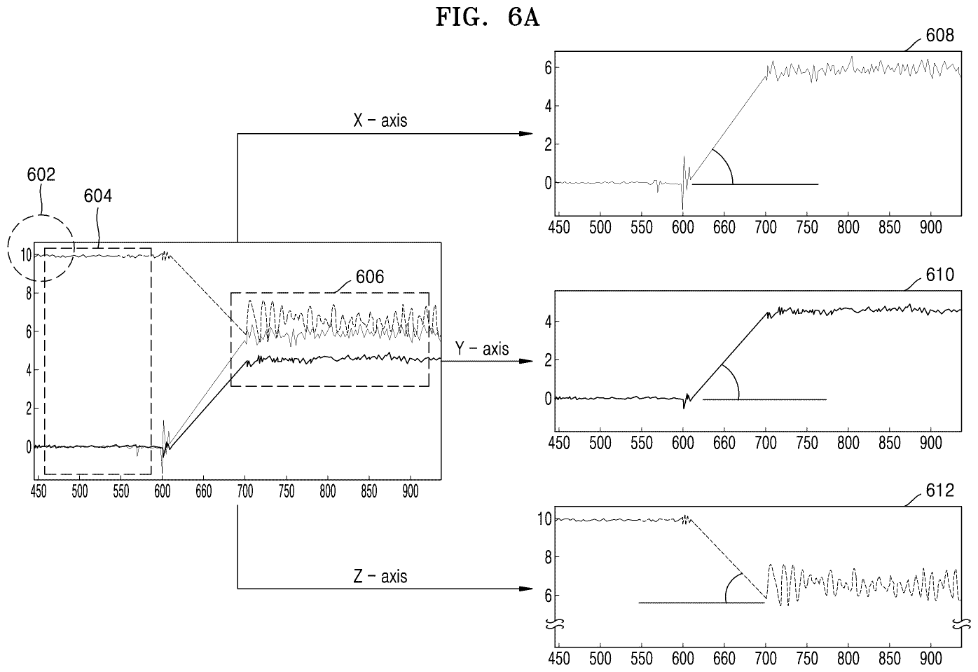

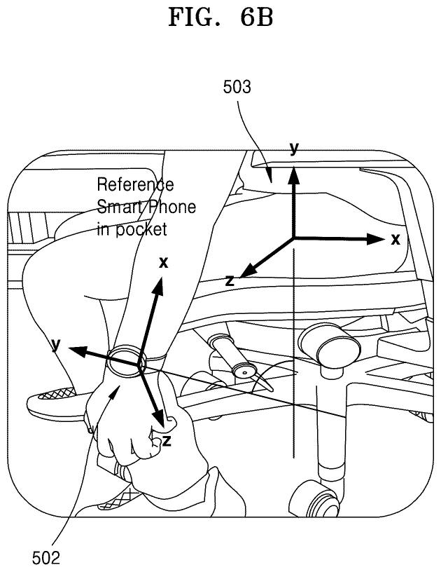

FIGS. 6A and 6B illustrate angle calculation by a hand angle calculator unit of a system, according to an embodiment of the disclosure.

Referring to FIGS. 6A and 6B, the hand angle calculator unit 110 may analyze an impact of acceleration due to gravity (e.g., g=9.8 m/s2) on the received accelerometer data to accurately identify the tilt angle of the wearable device 502. In an embodiment, the impact of the acceleration due to gravity is highlighted on one of the axes with a dotted circle 602. The impact of the acceleration due to gravity on any axis may maximize the magnitude of the acceleration data of said axis. A dotted rectangle 604 may represent waveforms when the wearable is placed on a top of a flat surface. In general, the impact of gravity varies with a change in height and location of the wearable device 502, therefore, when the wearable device 502 is placed on a flat surface, the impact of the gravity may be easily removed by the hand angle calculator unit 110. However, if the wearable device 502 has an acceleration in multiple axes, then the hand angle calculator unit 110 may remove the impact of the gravity and calculate the tilt angle using the following Equation 10-16:

$${tan}^{- 1} ( \frac{Acc ( x )}{ω 1} ) \qquad Equation 10$$

$$ω1 = \sqrt{{Acc ( y )}^{2} + {Acc ( z )}^{2}} \qquad Equation 11$$

$${tan}^{- 1} ( \frac{Acc ( y )}{ω 2} ) \qquad Equation 12$$

$$ω2 = \sqrt{{Acc ( x )}^{2} + {Acc ( z )}^{2}} - \qquad Equation 13$$

$${tan}^{- 1} ( \frac{Acc ( z )}{ω 3} ) \qquad Equation 14$$

$$ω3 = \sqrt{{Acc ( x )}^{2} + {Acc ( y )}^{2}} \qquad Equation 15$$

$${cos}^{- 1} [ \frac{\vec{Angle of Smart Wearable} \cdot \vec{Angle of Smart Phone}}{\vec{Angle of Smart Wearable} \times \vec{Angle of Smart Phone}} ] \qquad Equation 16$$

Here, the equations 10-11 may correspond to equations required to eliminate/remove the impact of gravity on the x-axis. Similarly, the Equations 12-13 and the Equations 14-15 may be defined for y-axis and z-axis, respectively. The variables ω1, ω2, and ω3 may defined a refined/modified value at axes x, y, and z, respectively. Moreover, the Equation 16 may be used to calculate the angle with the wearable device 102 and the mobile device 103 using the refined/modified variables.

Further, a dotted box 606 may represent waveforms when the wearable device 502 is in action, for example, the user wearing the wearable device 502 is scratching.

Three graphs 608, 610, and 612 illustrate a scenario when device moved from ideal to leg. The graph 608 may represent the waveform of change in acceleration that position in the x-axis. The graph 610 may represent the waveform of change in acceleration that position in the y-axis. The graph 612 may represent the waveform of change in acceleration that position in the z-axis.

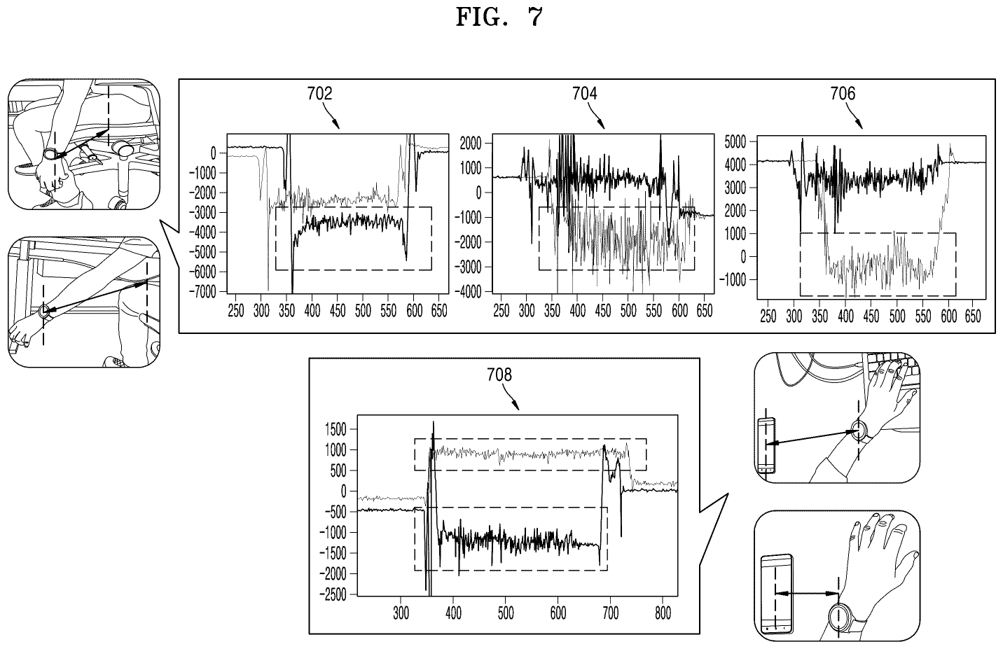

FIG. 7 illustrates the identification of potential scratching patterns by a user movement and hand posture identification unit of a system, according to an embodiment of the disclosure.

Referring to FIG. 7, the user movement and hand posture identification unit 112 may be configured to compare the determined distance and angle by the distance calculator unit 108 and the hand angle calculator unit 110, respectively with corresponding threshold values to determine whether the identified hand movement and/or hand posture of the user can correspond to a potential scratching/itching pattern. The user movement and hand posture identification unit 112 may use Table 2 below to classify the user's hand movement and/or hand posture as the potential scratching/itching pattern:

TABLE 2

| Distance | Angle | Potential Pattern |
|---|---|---|
| α < β | θ < ω | Not Potential |
| α < β | θ > ω | Not Potential |
| α > β | θ < ω | Not Potential |
| α > β | θ > ω | Potential |

Here, α may correspond to the distance between the wearable device 502 and the mobile device 503;

β may correspond to a threshold distance between the wearable device 502 and the mobile device 503;

θ may correspond to an angle between the wearable device 502 and the mobile device 503; and

ω may correspond to a threshold angle between the wearable device 502 and the mobile device 503. In an embodiment, the waveforms 702-706 may correspond to false cases detected by the angle with the same distance where the hand movement and/or the hand posture do not correspond to a potential scratching/itching pattern. The waveform 708 may correspond to a valid possibility based on the distance and the angle where the hand movement and/or the hand posture relate to a potential scratching/itching pattern.

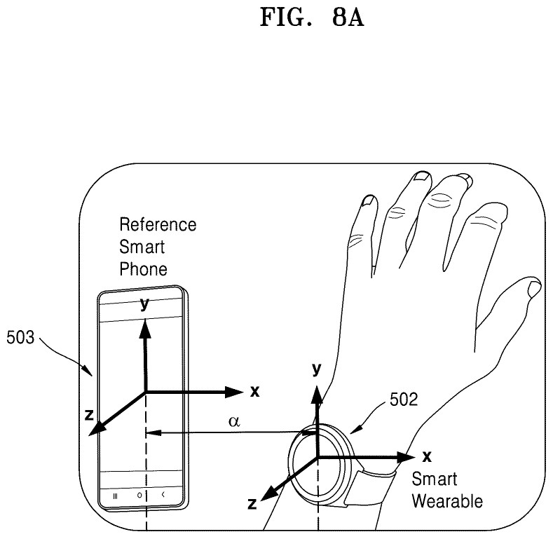

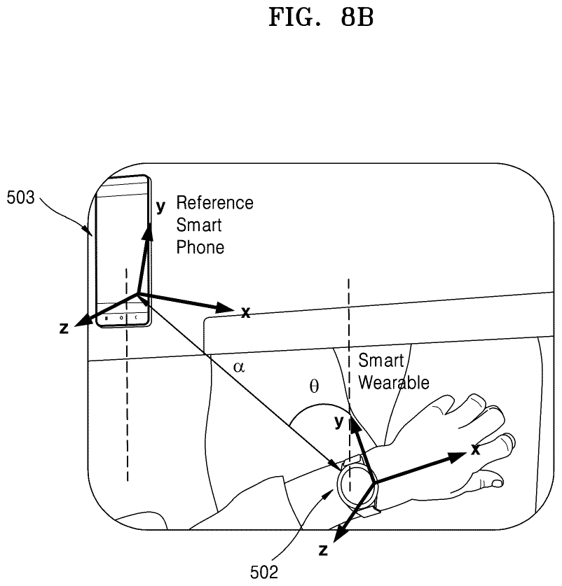

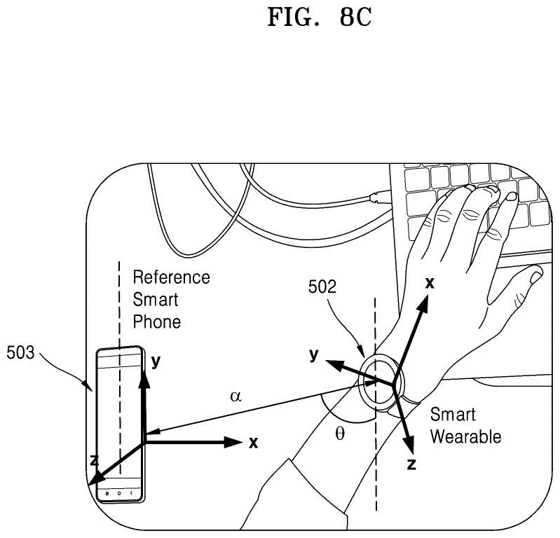

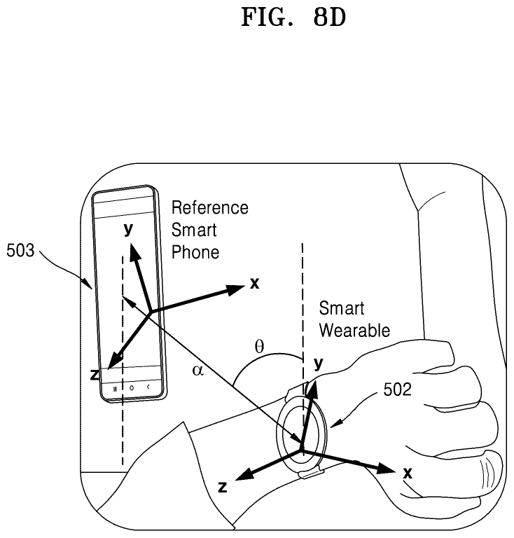

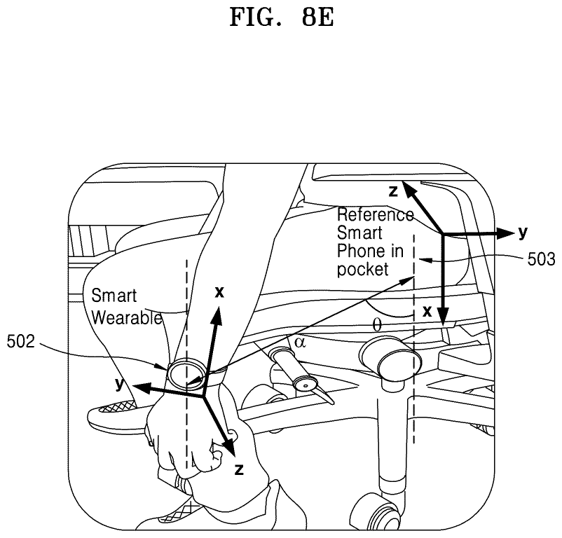

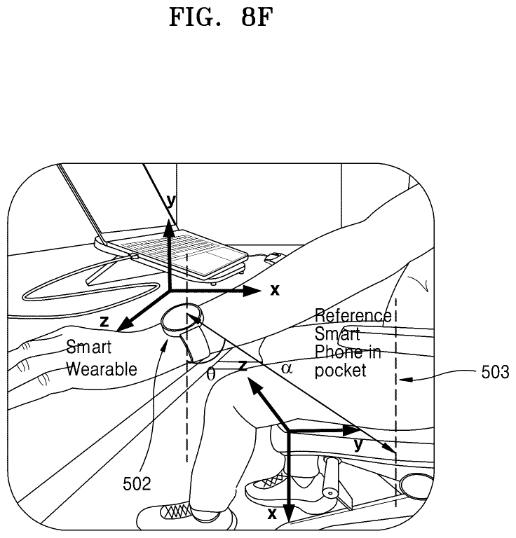

FIGS. 8A, 8B, 8C, 8D, 8E and 8F illustrates various scenarios of identification of the hand movement and/or the hand posture, according to an embodiment of the disclosure.

FIG. 8A illustrates a scenario when a user has the mobile device 503 that is placed on a table and the wearable device 502 is also placed on the table and is in rest position. In a scenario, the user movement and hand posture identification unit 112 may identify that the distance and the angle between the mobile device 503 and the wearable device 502 are within the corresponding threshold values, therefore if there is any hand movement and/or change in the hand posture, the hand movement and/or the change in the hand posture may relate to potential scratching.

FIG. 8B illustrates a scenario where the mobile device 503 may be placed on the table and the user is itching on the upper portion of the thighs. Here, the user movement and hand posture identification unit 112 may identify that the distance and the angle between the mobile device 503 and the wearable device 502 are within the corresponding threshold values, therefore, the scenario may be identified as a valid scenario of the potential scratching.

FIG. 8C illustrates a scenario where the mobile device 503 may be placed on the table and the user wearing the wearable device 502 is typing on a laptop placed on the table. Here, the identified distance may be within the predefined threshold, however, based on the identified angle, the user movement and hand posture identification unit 112 may consider the scenario as a false case.

FIG. 8D illustrates a scenario where the mobile device 503 may be placed on the table and the user wearing the wearable device 502 is scratching his other hand. Here, based on the valid distance between the mobile device 503 and the wearable device 502, and the hand movement, the user movement and hand posture identification unit 112 may consider the scenario as a valid scenario of the potential scratching.

FIG. 8E illustrates a scenario where the mobile device 503 is within a pocket of the user and the user wearing the wearable device 502 is scratching his leg. Here, based on a valid distance between the mobile device 503 and the wearable device 502, and a rapid change in the distance in the downward axis, the user movement and hand posture identification unit 112 may consider the scenario as a valid scenario of the potential scratching.

FIG. 8F illustrates a scenario where the mobile device 503 is within a pocket of the user and the user wearing the wearable device 502 is doing some hand movement outside a body range, therefore the user movement and hand posture identification unit 112 may consider the scenario as a false case of scratching.

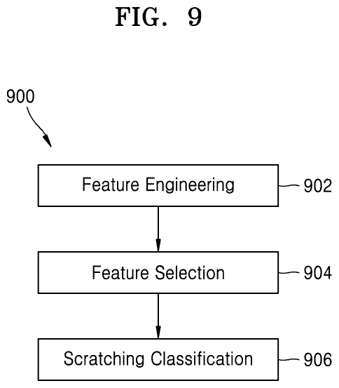

FIG. 9 illustrates a process flow of operations of a scratching signature detector unit of a system, according to an embodiment of the disclosure.

Referring to FIG. 9, at operation 902, the scratching signature detector unit 116 may perform feature engineering (e.g., calculate mathematical features corresponding to features/parameters of the potential scratching scenarios). At operation 904, the scratching signature detector unit 116 may perform a feature selection to select one or more mathematical features to be considered for identifying the scratching signature corresponding to the identified hand movement and/or the hand posture of the user. At operation 906, the scratching signature detector unit 116 may classify the identified hand movement and/or the hand posture of the user as one of the scratching signature or the non-scratching signature. The scratching signature detector unit 116 may process the data in the form of 20 s window having 50% overlapping for which a minimum 80% of similarity between features is required to classify the identified hand movement and/or the hand posture of the user as the scratching signature.

Some non-limiting example of the mathematical features utilized by the scratching signature detector unit 116 has been provided in Table 3 below:

TABLE 3

| Feature Name | About Feature | Mathematical Description |
|---|---|---|
| Mean for Acc(x), Acc(y), Acc(z), Co | Mean is average of all numbers in given window of | $Mean = \frac{\sum ( All values in window )}{Total values in window}$ |
| relations of Acc | data. | |
| (x, y, z) | | |
| Standard deviation for Acc(x), Acc(y), | It is measure of how much realization of | $Standard Deviation = \sqrt{\frac{\sum {v}^{2}}{window size} - {( \frac{\sum v}{window size} )}^{2} )}$ |
| Acc(z), Co | random variables | |
| relations of Acc | are dispersed | v = Value of data in given window |
| (x, y, z) | around its mean | |
| | in given size of | |
| | data window. | |
| PTP for Acc(x), | PTP is peak to | PTP = Max (Acc (axis variable)) − Min (Acc (axis variable)) |
| Acc(y), Acc(z), | peak range | |
| Co relations of | between min and | |
| Acc (x, y, z) | max values in | |
| | window size. | |
| RMS for Acc(x), Acc(y), Acc(z), Co relations of | RMS is root mean square which | $RMS = \sqrt{\frac{1}{Size of Window} \sum {( Each accelerometer ( axis variable ) )}^{2}}$ |
| Acc (x, y, z) | accelerometer | |
| | data which | |
| | represents | |
| | energy of given | |
| | signal in window | |
| IQR for Acc(x), Acc(y), Acc(z), Co relations of Acc (x, y, z) | IQR is interquartile range which measures the | ${1}^{st} IQR = \frac{Size of acc data window + 1}{4} for 1 st quartile and 3 rd quartile$ 3^{rd} IQR = 3*(1^{st} IQR) and IQR = 3^{rd} IQR − 1^{st} IQR |
| | spread of | |
| | accelerometer | |
| | data around | |
| | given % age. | |
| Permutation Entropy for Acc(x), Acc(y), | It is complexity of accelerometer data via phase | $H ( P ) = {\sum }_{i = 1}^{WIndow size !} value ( i ) * log \frac{1}{value ( i )}$ |
| Acc(z), Acc (x, | space | |
| y, z) | reconstruction | |
| Singular value decomposition entropy for Acc(x), Acc(y), | SVD represents the number of eigen vector that are needed for an | $SVD = - {\sum }_{1}^{M} \overline{σ} {log}_{2} \overline{σ} where \overline{σ} = \frac{σ}{{\sum }_{1}^{M} σ} and σ is singular spectrum of$ accelerometer data in sliding window. |
| Acc(z), Acc (x, | adequate | |
| y, z) | accelerometer | |
| | window size | |

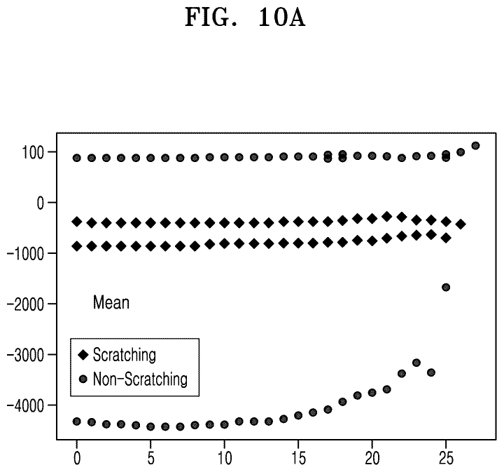

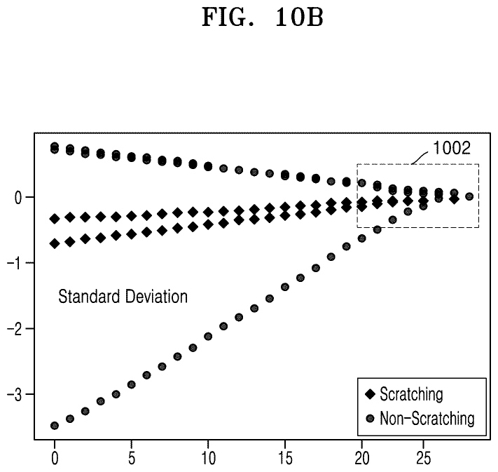

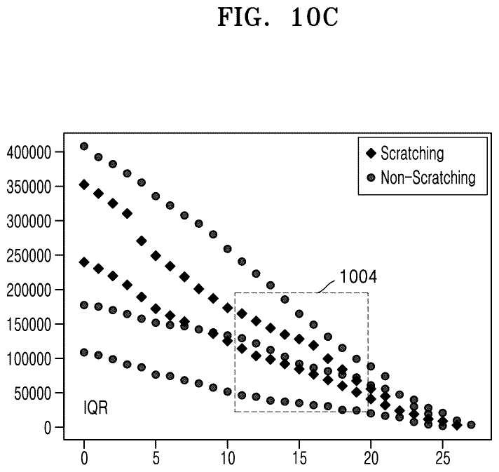

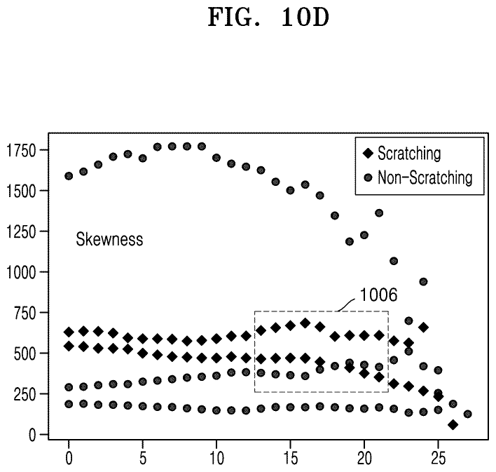

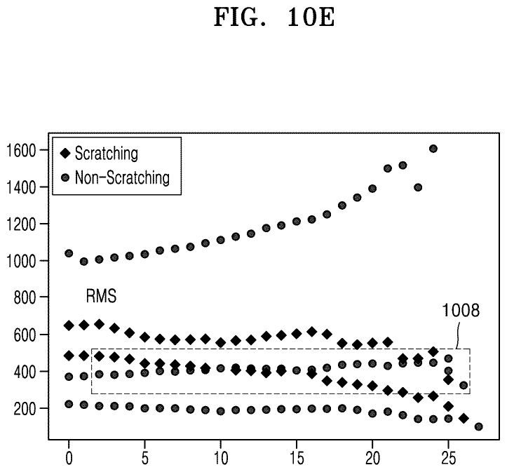

FIGS. 10A, 10B, 10C, 10D, and 10E illustrate graphical representations corresponding to various mathematical features used by a scratching signature detector unit, according to an embodiment of the disclosure. In particular, FIG. 10A may correspond to a mathematical feature “mean”, FIG. 10B may correspond to a mathematical feature “standard deviation”, FIG. 10C may correspond to a mathematical feature “IQR”, FIG. 10D may correspond to the mathematical feature “skewness”, and FIG. 10E may correspond to the mathematical feature “Root Mean Square (RMS)”.

Referring to FIGS. 10A-10E, the dotted region 1002-1008 may represent the overlapping of non-scratching movements with the potential scratching movements. The scratching signature detector unit 116 may eliminate said overlapping regions by selecting specific features and applying a classifier. The various graphical representations illustrated in FIG. 10A-10E may represent the classification of the potential scratching movements from the non-scratching movements.

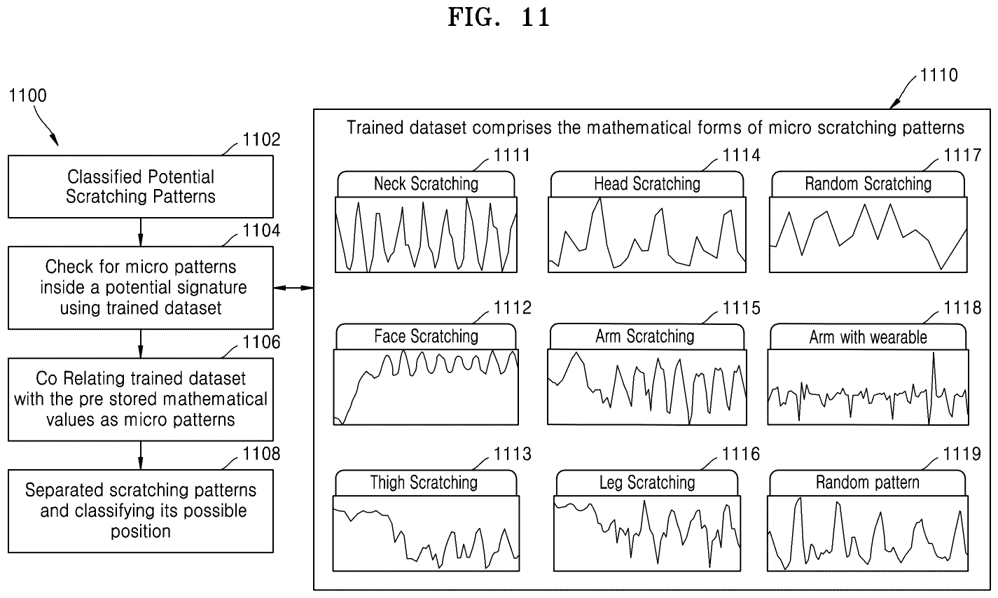

FIG. 11 illustrates a process flow 1100 of operations of a signature analyzer unit, according to an embodiment of the disclosure.

Referring to FIG. 11, at operation 1102, the signature analyzer unit 118 may classify potential scratching patterns in at least one type of scratching. At operation 1104, the signature analyzer unit 118 analyzes the micro-pattern within the potential scratching patterns with respect to a trained dataset 1110. The trained dataset 1110 may be stored in the second trained database 130. At operation 1106, the signature analyzer unit 118 may co-relate the trained dataset 1110 with the pre-stored mathematical values as micro patterns to classify the potential scratching patterns. At operation 1108, the signature analyzer unit 118 may separate the potential scratching patterns from the non-scratching patterns and classify the potential scratching patterns based on a position of the scratching on the user's body.

The trained dataset of neck scratching 1111 may include at least one of the mathematical values: Mean (3113-3637), std (87-1395), ptp (307-6073), Skew (−0.79-0.56), kutosis (−1.48-2.18), rms (3180-3655), iqr (97-2307), Perm (0.944-1), and Svd (0.13-0.8). The trained dataset of face scratching 1112 may include at least one of the mathematical values: Mean (−828-675), std (221-996), ptp (861-6190), Skew (−1.25-0.98), kutosis (−1.03-1.08), rms (224-1154), iqr (207-1379), Perm (0.88-0.99), and Svd (0.95-0.99). The trained dataset of thigh scratching 1113 may include at least one of the mathematical values: Mean (−3613-2807), std (39-3077), Skew (−1.44-17318), kutosis (−1.48-1.87), iqr (48-5168), and Perm (0-1). The trained dataset of head scratching 1114 may include at least one of the mathematical values: Mean (−1815-2363), std (269-1546), ptp (538-6439), Skew (−0.37-1.11), kutosis (−2-3.94), rms (737.97-2378.26), iqr (269-2627), Perm (0.95-1), and Svd (0.68-0.993). The trained dataset of arm scratching 1115 may include at least one of the mathematical values: Mean (−3836-1516), std (299-1520), ptp (919-11408), Skew (−2.2-0.84), kutosis (−1.23-10.11), rms (972-3986), iqr (412-1614), Perm (0.97-1), and Svd (0.53-0.99). The trained dataset of leg scratching 1116 may include at least one of the mathematical values: Mean (−420-970), std (38-136), Skew (−2.3-0.2), kutosis (−2-7.9), iqr (38-166), and Perm (0.91-0.99). The trained dataset of random scratching 1117 may include at least one of the mathematical values: Mean (323-2793), std (230-1475), ptp (854-5377), Skew (−0.48-0.4), kutosis (−1.14-0.95), rms (447-2891), iqr (320-2175), Perm (0.97-1), and Svd (0.43-0.98). The trained dataset of arm with wearable 1118 may include at least one of the mathematical values: Mean (−3361-144), std (109-2052), ptp (299-26072), Skew (−1.42-2.91), kutosis (−1.5-16.3), rms (828-3436), iqr (136.25-2710), Perm (0.95-0.99), and Svd (0.3-0.99). The trained dataset of random pattern 1119 may include at least one of the mathematical values: Mean (−4117-3453), std (421-1042), Skew (−1.09-0.06), kutosis (−1.39-0.81), iqr (631-1712), and Perm (0.98-1).

The signature analyzer unit 118 may process a window size of n in the accelerometer data, (e.g., n=20) (e.g., the signature analyzer unit 118 may have 20 data points to classify into some m classes of scratching e.g., scratching of head, neck, legs, thigs, arms, hands, face, etc.) The signature analyzer unit 118 may select some features that can be fed into an ML model to get a weak classifier. The system 100 may generate an ML model to minimize Mean Squared Error (MSE) in between scratching classes. According to an embodiment of the disclosure, the signature analyzer unit 118 may utilize a custom micro pattern classifier via decision trees boosted with a gradient to process continuous time series data of scratching.

The ML model may be generated with the loss function provided by the following Equation 17:

$$Loss function for scratching = arg {min}_{\begin{matrix}Scratching \\ Predicted \\ value\end{matrix}} {\sum }_{i = 1}^{20} Loss Func ( y , predicted scratching ( γ ) ) \qquad Equation . 17$$

Here, the “arg min” represents that the system 100 needs to identify the scratching prediction threshold for which the loss function is minimum. The loss function for scratching may help to determine the rate of error between an output of the ML model and an actual scratching position. The loss function may indicate the efficiency of the ML model to handle different scratching scenarios on different positions of bodies.

$$Loss function = \frac{1}{20} {\sum }_{i = 1}^{20} {( y - predicted scratching ( γ ) )}^{2} \qquad Equation 18$$

$$\frac{d Loss}{d Scratching} = \frac{1}{2} ( {\sum }_{i = 1}^{20} ( y - predicted scratching ( γ ) ) \qquad Equation 19$$

A scratching threshold value used for co-relation and identification of the skin diseases may be defined according to Equation 20:

$$Scratching threshold = \frac{1}{2} {( 4155.85 - γ )}^{2} + \frac{1}{2} {( 4172.84 - γ )}^{2} + \frac{1}{2} {( 4134.46 - γ )}^{2} + \frac{1}{2} {( 4125.02 - γ )}^{2} + \frac{1}{2} {( 4219.81 - γ )}^{2} \qquad Equation 20$$

Predicted scratching threshold=4141.596

The predicted scratching threshold value of 4141.596 may vary based on the user's wearable devices.

The system 100 may calculate a pseudo residual that may indicate a distance/difference between the output of the predicted scratching position by the ML model and the actual values. The scratching residual may indicate the distance between the position of the predicted scratching value and the actual scratching position. The scratching residual may be defined by the following Equations 21-25:

$$Scratching Residual = - ( \frac{\partial Scratching Loss ( y , Loss Function )}{\partial Loss Function} ) \qquad Equation 21$$

$$L = - {\sum }_{i = 1}^{20} y log ( p ) + ( 1 - p ) log ( 1 - p ) \qquad Equation 22$$

$$L = - y * ( \frac{log ( p )}{log ( i - p )} ) - log ( 1 - p ) = - y * log ( \frac{p}{1 - p} ) = - y * log ( odds ) - log ( 1 - p ) \qquad Equation 23$$

$$L = - y * log ( odds ) + log ( 1 + {e}^{log ( odds )} ) \qquad Equation 24$$

$$\frac{dL}{d ( log ( odds ) )} = - y + \frac{{e}^{log ( odds )}}{1 + {e}^{log ( odds )}} \qquad Equation 25$$

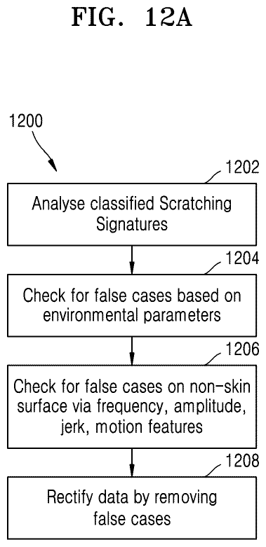

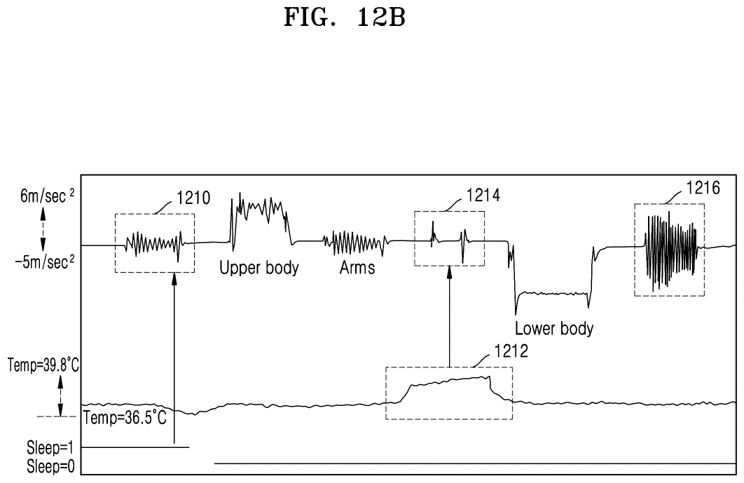

FIG. 12A illustrates a process flow 1200 of operations of a false scratching eliminator unit, according to an embodiment of the disclosure.

FIG. 12B illustrates a graphical representation of waveforms representing scratching on a skin surface or a non-skin surface, according to an embodiment of the disclosure. For the sake of brevity, FIGS. 12A-12B have been explained in conjunction with each other.

Referring to FIGS. 12A and 12B, at operation 1202, the false scratching eliminator unit 120 may analyze the classified scratching signatures. At operation 1204, the false scratching eliminator unit 120 may check for false cases among the classified scratching signatures based on environmental parameters. At operation 1206, the false scratching eliminator unit 120 may check for false cases among the classified scratching signatures on non-skin surfaces based on features/parameters such as, but not limited to, frequency, amplitude, jerk, and other motion features. For example, skin and body have some frication due to which force needed to scratch on the body will be more than non-skin surfaces, thus based on the determined frequency of the scratching, the false scratching eliminator unit 120 may classify the scratching as being done on skin-surface or non-skin surface. The skin may be easily deformed at any place while scratching which will be easily visible in the amplitude component. The skin is not rigid, so the skin may produce more random motion than the non-skin surface, which may be identified from the motion components. In the case of scratching, the jerk may be defined as a rate of change of acceleration with respect to time. Since, skin is deformable so changes in acceleration and de-acceleration e.g., jerks in acceleration will be more frequent and greater in number than non-skin surfaces. Thus, the false scratching eliminator unit 120 may identify the false cases based on the above-mentioned parameters/features.

At operation 1208, the false scratching eliminator unit 120 may rectify data by removing false cases. All the false cases as also illustrated in FIG. 12B may be filtered out and marked as non-deteriorating for skin. The waveforms may correspond to the received accelerometer data. A dotted box 1210 may represent the waveform that scratching pattern during sleep (e.g. ‘Sleep=1’ means the user is sleeping and ‘Sleep=0’ means the user is in a wake state) which can be an indication of disease if frequency increases over time. A dotted box 1212 may represent the waveform of change in temperature. A dotted box 1214 may represent the waveform when itching due to change in environmental conditions (e.g. temperature, humidity) which can be skipped/classified as non-deteriorating for skin. A dotted box 1216 may represent the waveform of a scratching pattern on a non-skin surface which can/can't be scratching, the pattern is similar but somewhat different and can be checked in the upcoming module. The system 100 may evaluate the scratching patterns which are similar to scratching on the skin-surface and may be deteriorating for the skin.

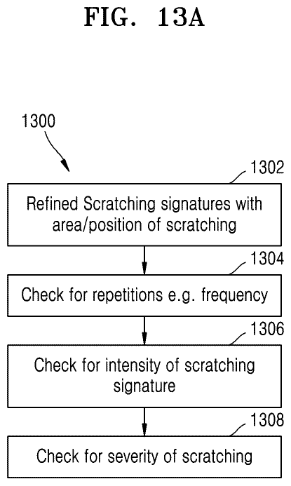

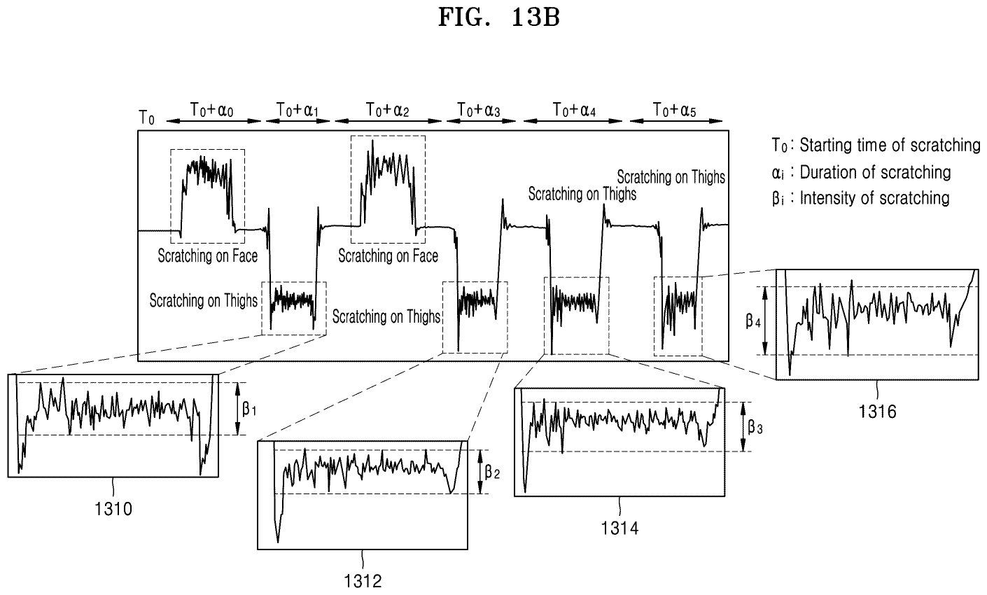

FIG. 13A illustrates a process flow 1300 of operations of a feature generator unit, according to an embodiment of the disclosure.

FIG. 13B illustrates a graphical representation of waveforms representing a level of scratching and severity, according to an embodiment of the disclosure. For the sake of brevity, FIGS. 13A and 13B have been explained in conjunction with each other.

Referring to FIGS. 13A and 13B, the feature generator unit 126 may be configured to generate features for classified scratching signatures to check contributions in skin deterioration like frequency (number of times the user scratches), intensity (how intense is scratching signature), and severity of scratching. At operation 1302, the feature generator unit 126 may refine the identified scratching signature based on the area/position of the scratching. At operation 1304, the feature generator unit 126 may identify the repetition/frequency of the scratching signatures. At operation 1306, the feature generator unit 126 may determine the intensity of the scratching signatures. At operation 1308, the feature generator unit 126 may determine the severity of the scratching signatures. FIG. 13B illustrates waveforms representing the frequency and intensity of the scratching signatures utilized to identify the severity of the scratching by the feature generator unit 126. The waveforms may correspond to the received accelerometer data. Two dotted boxes may represent the waveforms of scratching on face which may not contribute to skin deterioration as it's not severe. A graph 1310 may represent the waveform of 1st time scratching on thighs, which may correspond to the data: average values (−258.164), number of peaks (14), and average distance between peaks (8.76). A graph 1312 may represent the waveform of 2nd time scratching on thighs, which may correspond to the data: average values (−419.580), number of peaks (11), and average distance between peaks (9.7). A graph 1314 may represent the waveform of 3rd time scratching on thighs, which may correspond to the data: average values (−421.271), number of peaks (14), and average distance between peaks (9.3). A graph 1316 may represent the waveform of 4th time scratching on thighs, which may correspond to the data: average values (−423.544), number of peaks (9), and average distance between peaks (10.2). For instance, the user scratching frequently on the lower body with similar intensity as done previously over a period of time may indication the presence of some skin diseases in the user.

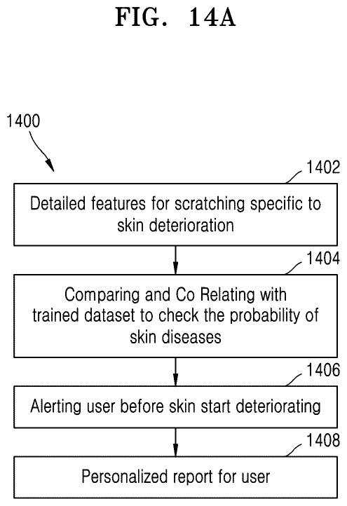

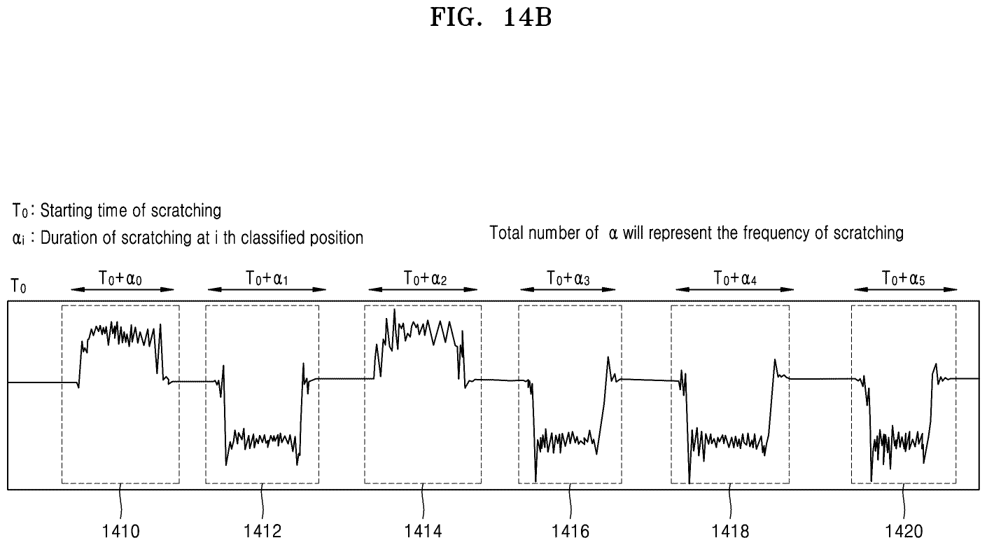

FIG. 14A illustrates a process flow 1400 of operations of a scratching evaluator unit, according to an embodiment of the disclosure.

FIG. 14B illustrates a graphical representation of waveforms representing scratching on different body parts with associated frequencies, according to an embodiment of the disclosure. For the sake of brevity, FIGS. 14A and 14B have been explained in conjunction with each other.

Referring to FIGS. 14A and 14B, the scratching evaluator unit 128 may be configured to analyze the features specific to deterioration like frequency, intensity, and severity by co-relating with a trained dataset to predict any risks for skin deterioration and probability of skin disease in the user. The trained dataset may be stored in the second trained database 130. The skin diseases may include, but are not limited to, dry skin (xerosis), psoriasis, scabies, parasites, burns, scars, eczema/atopic dermatitis, contact dermatitis, seborrheic dermatitis, dyshidrotic dermatitis, neuro dermatitis, nummular dermatitis, periorificial dermatitis, stasis dermatitis, excoriation disorder, nocturnal Pruritus, and the like.

At operation 1402, the scratching evaluator unit 128 may analyze the features for scratching specific to skin deterioration conditions. At operation 1404, the scratching evaluator unit 128 may compare and co-relate the features with the trained dataset to check the probability of skin diseases. At operation 1406, the scratching evaluator unit 128 may generate the dynamic alerts 134 to alert the user before the skin starts deteriorating. At operation 1408, the scratching evaluator unit 128 may generate the personalized report 132 for the user. FIG. 14B illustrates waveforms representing scratching on different body parts along with assigned weights, frequency, counts, and probability of skin disease. The waveforms may correspond to the received accelerometer data. The waveform of scratching on face 1410 may represent ‘frequency count=1’ and ‘scratching count=1’. As this is different from previous, so weightage toward skin deterioration must decrease (e.g. weightage for face=100%). The waveform of scratching on thighs 1412 may represent ‘frequency count=1’ and ‘scratching count=2’. As this is different from previous, so weightage toward skin deterioration must decrease (e.g. weightage for face=50%, weightage for thighs=50%). The waveform of scratching on face 1414 may represent ‘frequency count=2’ and ‘scratching count=3’. As this is different from previous but similar to recently occurred pattern, so weightage toward skin deterioration must change (e.g. weightage for face=70%, weightage for thighs=30%). The waveform of scratching on thighs 1416 may represent ‘frequency count=2’ and ‘scratching count=4’. As this is different from previous but similar to recently occurred pattern, so weightage toward skin deterioration must change (e.g. weightage for face=50%, weightage for thighs=50%). The waveform of scratching on thighs 1418 may represent ‘frequency count=3’ and ‘scratching count=5’. As this is similar to previously occurred pattern, so weightage toward skin deterioration must change (e.g. weightage for face=40%, weightage for thighs=60%). The waveform of scratching on thighs 1420 may represent ‘frequency count=4’ and ‘scratching count=6’. As this is similar to previously occurred pattern, so weightage toward skin deterioration must change (e.g. weightage for face=20%, weightage for thighs=80%).

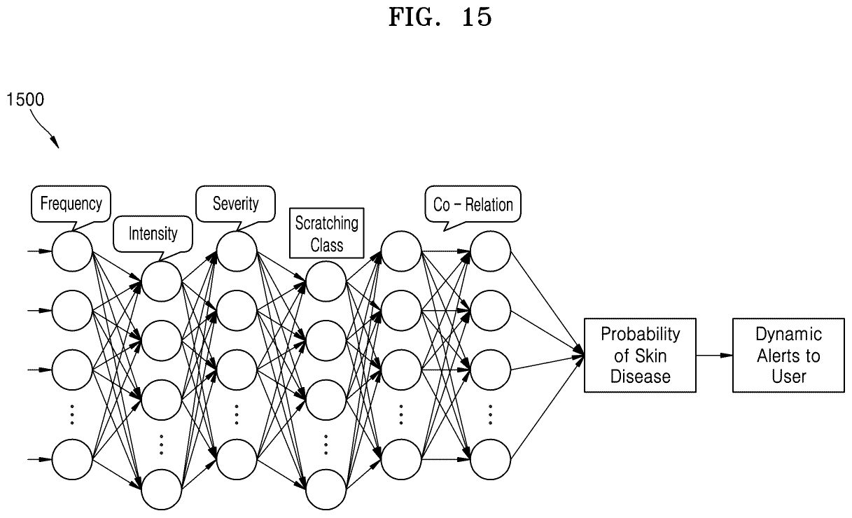

FIG. 15 illustrates a schematic representation of a machine learning (ML) model utilizing a trained dataset to predict the presence of skin disease in the user, according to an embodiment of the disclosure.

Referring to FIG. 15, the trained dataset as represented by the second trained database 130 in FIG. 1 may include mathematical forms of the features like frequency, intensity, severity, and associated relations with possible skin diseases. A non-limiting example of the trained dataset is represented using below Table 4:

TABLE 4

| Scratching | | | | |
|---|---|---|---|---|
| Body | Frequency | Intensity | Severity | Possible |
| Part | Threshold | Threshold | Threshold | Diseases |
| Skin - red, | 5 | 4 | low | Atopic |
| inflamed, | | | | Dermatitis |
| dry, bumpy | | | | (Eczema) |
| Scalp - yellow | 5 | 3 | low | Seborrheic |
| patches around | | | | Dermatitis |
| red rashes | | | | |
| Edges - Fingers, | 7 | 5 | Medium | Dyshidrotic |
| palms, | | | | Dermatitis |
| toes, feet | | | | |
| Nerves- Red, | 10 | 7 | high | Neuro Dermatitis |
| Irritation | | | | |
| Skin - dry, open | 6 | 3 | medium | Nummular |
| sores | | | | Dermatitis |
| Face/Lower | 5 | 3 | high | Periorificial |
| Body - | | | | Dermatitis |
| Mouth, Eyes, | | | | |
| Nose | | | | |
| Ankles | 4 | 3 | medium | Stasis Dermatitis |
| Night Scratching | 4 | 4 | high | Nocturnal |
| | | | | Pruritus |

The ML model 1500 may be represented using below Equation(s) 26-27:

$$F ( Scratching ) = {\sum }_{i = 0}^{i = Total Scratching Count} α i * {i}^{th} Scratching * Weight ( i ) = \sum α i * {i}^{th} Scratching * Weight ( i ) \qquad Equation 26$$

$$F ( Skin Diseases ) = F ( Scratching ) [ α n ] \qquad Equation 27$$

Here, a number of α may represent the frequency of scratching. Further, Weight(i) may increase if similar scratching patterns repeats in window and decrease if not.

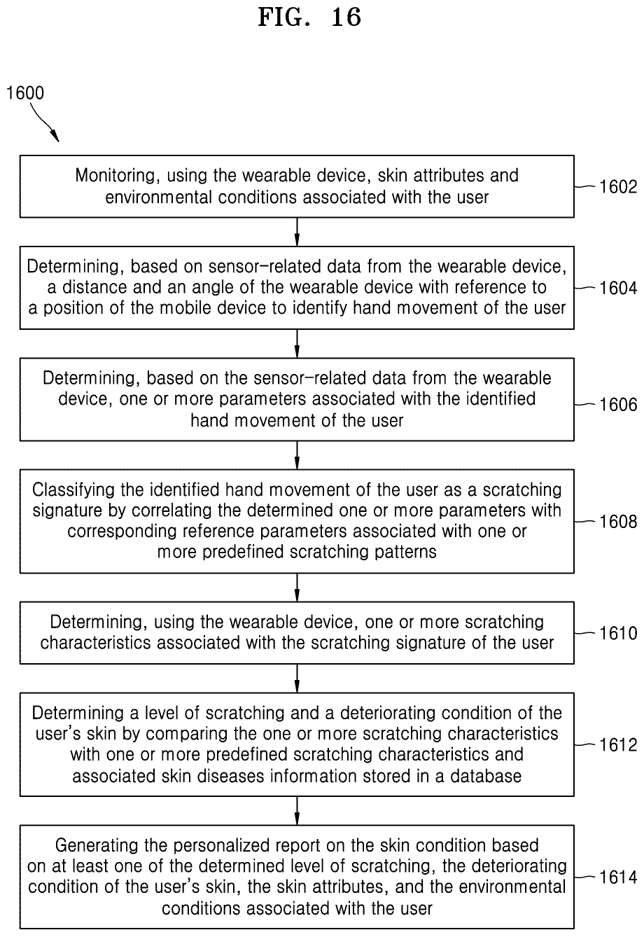

FIG. 16 illustrates a flow chart of a method 1600 for generating a personalized report on the skin condition of a user, according to an embodiment of the disclosure.

Referring to FIG. 16, the method 1600 may be implemented by the one or more components of the system 100.

At operation 1602, the method 1600 may include monitoring, using the wearable device 102, skin attributes, and environmental conditions associated with the user.

At operation 1604, the method 1600 may include determining, based on sensor-related data from at least one accelerometer sensor of the wearable device 102, a distance, and an angle of the wearable device 102 with reference to a position of the mobile device 103 to identify hand movement of the user.

At operation 1606, the method 1600 may include determining, based on the sensor-related data from at least one accelerometer sensor, one or more parameters associated with the identified hand movement of the user.

At operation 1608, the method 1600 may include classifying the identified hand movement of the user as a scratching signature by correlating the determined one or more parameters with corresponding reference parameters associated with one or more predefined scratching patterns.

At operation 1610, the method 1600 may include determining, using the wearable device 102, one or more scratching characteristics associated with the scratching signature of the user.

At operation 1612, the method 1600 may include determining a level of scratching and deteriorating condition of the user's skin by comparing the one or more scratching characteristics with one or more predefined scratching characteristics and associated skin diseases information stored in a database.

At operation 1614, the method 1600 may include generating the personalized report 132 on the skin condition based at least on the determined level of scratching, the deteriorating condition of the user's skin, the skin attributes, and the environmental conditions associated with the user.

An embodiment as discussed above is example in nature and the method 1600 may include any additional step to perform the desired objective of the disclosure. Further, the steps of the method 1600 may be performed in any suitable manner in order to achieve the desired advantages.

Thus, the disclosure may enable the user to effectively detect the presence of skin disease before the skin starts deteriorating. In particular, the disclosure may provide a simple and effective technique to identify scratching patterns using the accelerometer sensor data embedded within the wearable device and/or the mobile device of the user.

It will be appreciated that an embodiment of the disclosure according to the claims and description in the specification can be realized in the form of hardware, software or a combination of hardware and software.

Any such software may be stored in computer readable storage media (e.g. a non-transitory computer-readable storage media). The computer readable storage media store one or more computer programs (software modules), the one or more computer programs include computer-executable instructions that, when executed by one or more processors of an electronic device, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage device and storage medium is an embodiment of machine-readable storage (e.g. a non-transitory machine-readable storage) that are suitable for storing a computer program or computer programs comprising instructions that, when executed, implement an embodiment of the disclosure. Accordingly, an embodiment provide a program comprising code for implementing apparatus or a method as claimed in any one of the claims of this specification and a machine-readable storage storing such a program.

While the disclosure has been shown and describe with reference to an embodiment thereof, it will be understood by those skilled in the art that various changes in form and details may be made therein without departing from the spirit and scope of the disclosure as defined by the appended claims and their equivalents.

In an embodiment, the method, wherein the one or more scratching characteristics may include at least one of an area of scratching, a frequency of scratching, and an intensity of scratching.

In an embodiment, the method, wherein the skin attributes may include at least one of softness, a moisture level, and a dryness level of the user's skin.

In an embodiment, the method, wherein the environmental conditions may include at least one of a humidity level and a temperature associated with a surrounding of the user.

In an embodiment, the method, wherein the one or parameters associated with the identified hand movement of the user may include at least one of frequency, amplitude, motion, and jerks.

In an embodiment, the method, wherein, using the ML model, the identified hand movement of the user as the scratching signature may include comparing the determined distance and the angle of the wearable device with corresponding predefined thresholds. The method, wherein, using the ML model, the identified hand movement of the user as the scratching signature may include upon determining that the determined distance and the angle of the wearable device is less than the corresponding predefined thresholds, classifying the identified hand movement of the user as the scratching signature.

In an embodiment, the method, wherein upon determining that at least one of the determined distance and the angle of the wearable device is greater than the corresponding predefined thresholds, classifying the identified hand movement of the user as a non-scratching signature.

In an embodiment, the method, wherein the scratching signature may include at least one of neck scratching, head scratching, face scratching, arm scratching, thigh scratching, leg scratching, and random scratching.

In an embodiment, the method, wherein determining the deteriorating condition of the user's skin may include monitoring the identified hand movement for a predefined time duration. The method, wherein determining the deteriorating condition of the user's skin may include identifying one or more changes in the identified hand movement during the predefined time duration. The method, wherein determining the deteriorating condition of the user's skin may include correlating the identified one or more changes with the environmental conditions. The method, wherein determining the deteriorating condition of the user's skin may include determining the deteriorating condition of the user's skin based on the correlation.

In an embodiment, the method may include generating, via at least one of the wearable device or the mobile device of the user, an alert based on the generated personalized report.

In an embodiment, the system, wherein the one or more scratching characteristics may include at least one of an area of scratching, a frequency of scratching, and an intensity of scratching.

In an embodiment, the system, wherein the skin attributes may include at least one of softness, a moisture level, and a dryness level of the user's skin.

In an embodiment, the system, wherein the environmental conditions may include at least one of a humidity level and a temperature associated with a surrounding of the user.

In an embodiment, the system, wherein the one or more parameters associated with the identified hand movement of the user may include at least one of frequency, amplitude, motion, and jerks.

In an embodiment, the system, wherein to classify, using the ML model, the identified hand movement of the user as the scratching signature, the one or more computer programs further include instructions that, when executed by the one or more processors, cause the system to compare the determined distance and the angle of the wearable device with corresponding predefined thresholds. The one or more processors cause the system to upon determining that the determined distance and the angle of the wearable device is less than the corresponding predefined thresholds, classify the identified hand movement of the user as the scratching signature.

In an embodiment, the system, wherein upon determining that at least one of the determined distance and the angle of the wearable device is greater than the corresponding predefined thresholds, the one or more computer programs further include instructions that, when executed by the one or more processors, cause the system to classify the identified hand movement of the user as a non-scratching signature.

In an embodiment, the system, wherein the scratching signature may include at least one of neck scratching, head scratching, face scratching, arm scratching, thigh scratching, leg scratching, and random scratching.

In an embodiment, the system, wherein to determine the deteriorating condition of the user's skin, the one or more computer programs further include instructions that, when executed by the one or more processors, cause the system to monitor the identified hand movement for a predefined time duration. The one or more processors cause the system to identify one or more changes in the identified hand movement during the predefined time duration. The one or more processors cause the system to correlate the identified one or more changes with the environmental conditions. The one or more processors cause the system to determine the deteriorating condition of the user's skin based on the correlation.

In an embodiment, the system, wherein the one or more computer programs further include instructions that, when executed by the one or more processors, cause the system to generate, via at least one of the wearable device or the mobile device of the user, an alert based on the generated personalized report.

In an embodiment, the system, wherein the wearable device may include a transceiver configured to communicate with the mobile device. The system, wherein the wearable device may include the at least one acceleration sensor. The system, wherein the wearable device may include the memory. The system, wherein the wearable device may include the one or more processors, wherein the one or more processors are communicatively coupled to the transceiver, the at least one acceleration sensor, and the memory.

In an embodiment, the system, wherein the wearable device may include the at least one accelerometer sensor. The system, wherein the mobile device may include a transceiver configured to communicate with the wearable device. The system, wherein the mobile device may include the memory. The system, wherein the mobile device may include the one or more processors, wherein the one or more processors are communicatively coupled to the transceiver and the memory.

In an embodiment, the one or more computer-readable storage media, the operations may include generating, via at least one of the wearable device or the mobile device of the user, an alert based on the generated personalized report.

What is claimed is:

1. A method for generating a personalized report on skin condition of a user using a wearable device connected to a mobile device of the user, the method comprising:
monitoring, using the wearable device, skin attributes and environmental conditions associated with the user;
determining, based on sensor-related data from the wearable device, a distance and an angle of the wearable device with reference to a position of the mobile device to identify hand movement of the user;
determining, based on the sensor-related data from the wearable device, one or more parameters associated with the identified hand movement of the user;
classifying the identified hand movement of the user as a scratching signature by correlating the determined one or more parameters with corresponding reference parameters associated with one or more predefined scratching patterns;
determining, using the wearable device, one or more scratching characteristics associated with the scratching signature of the user;
determining a level of scratching and a deteriorating condition of the user's skin by comparing the one or more scratching characteristics with one or more predefined scratching characteristics and associated skin diseases information stored in a database; and
generating the personalized report on the skin condition based on at least one of the determined level of scratching, the deteriorating condition of the user's skin, the skin attributes, and the environmental conditions associated with the user.

2. The method as claimed in claim 1, wherein the one or more scratching characteristics comprises at least one of an area of scratching, a frequency of scratching, and an intensity of scratching.

3. The method as claimed in claim 1, wherein the skin attributes comprise at least one of softness, a moisture level, and a dryness level of the user's skin.

4. The method as claimed in claim 1, wherein the environmental conditions comprise at least one of a humidity level and a temperature associated with a surrounding of the user.

5. The method as claimed in claim 1, wherein the one or more parameters associated with the identified hand movement of the user comprises at least one of frequency, amplitude, motion, and jerks.

6. The method as claimed in claim 1, wherein classifying the identified hand movement of the user as the scratching signature comprises:
comparing the determined distance and the angle of the wearable device with corresponding predefined thresholds; and
upon determining that the determined distance and the angle of the wearable device is less than the corresponding predefined thresholds, classifying the identified hand movement of the user as the scratching signature.

7. The method as claimed in claim 1, wherein the scratching signature comprises at least one of neck scratching, head scratching, face scratching, arm scratching, thigh scratching, leg scratching, and random scratching.

8. The method as claimed in claim 1, wherein determining the deteriorating condition of the user's skin comprises:
monitoring the identified hand movement for a predefined time duration;
identifying one or more changes in the identified hand movement during the predefined time duration;
correlating the identified one or more changes with the environmental conditions; and
determining the deteriorating condition of the user's skin based on the correlation.

9. The method as claimed in claim 1, further comprising:
generating, via at least one of the wearable device or the mobile device of the user, an alert based on the generated personalized report.

10. A system for predicting skin deteriorating condition of a user using a wearable device connected to a mobile device of the user, the system comprising:
memory storing one or more computer programs; and
one or more processors communicably coupled with the memory,
wherein the one or more computer programs include computer-executable instructions that, when executed by the one or more processors, cause the system to:
monitor, using the wearable device, skin attributes and environmental conditions associated with the user,
determine, based on sensor-related data from the wearable device, a distance and an angle of the wearable device with reference to a position of the mobile device to identify hand movement of the user,
determine, based on the sensor-related data from the wearable device, one or more parameters associated with the identified hand movement of the user,
classify the identified hand movement of the user as a scratching signature by correlating the determined one or more parameters with corresponding reference parameters associated with one or more predefined scratching patterns,
determine, using the wearable device, one or more scratching characteristics associated with the scratching signature of the user,
determine a level of scratching and a deteriorating condition of the user's skin by comparing the one or more scratching characteristics with one or more predefined scratching characteristics and associated skin diseases information stored in a database, and
generate a personalized report on the skin condition based on at least one of the determined level of scratching, the deteriorating condition of the user's skin, the skin attributes, and the environmental conditions associated with the user.

11. The system as claimed in claim 10, wherein the one or more scratching characteristics comprises at least one of an area of scratching, a frequency of scratching, and an intensity of scratching.

12. The system as claimed in claim 10, wherein the skin attributes comprise at least one of softness, a moisture level, and a dryness level of the user's skin.

13. The system as claimed in claim 10, wherein the environmental conditions comprise at least one of a humidity level and a temperature associated with a surrounding of the user.

14. The system as claimed in claim 10, wherein the one or more parameters associated with the identified hand movement of the user comprises at least one of frequency, amplitude, motion, and jerks.

15. The system as claimed in claim 10, wherein to classify the identified hand movement of the user as the scratching signature, the one or more computer programs further include instructions that, when executed by the one or more processors, cause the system to:
compare the determined distance and the angle of the wearable device with corresponding predefined thresholds; and
upon determining that the determined distance and the angle of the wearable device is less than the corresponding predefined thresholds, classify the identified hand movement of the user as the scratching signature.

16. The system as claimed in claim 10, wherein the scratching signature comprises at least one of neck scratching, head scratching, face scratching, arm scratching, thigh scratching, leg scratching, and random scratching.

17. The system as claimed in claim 10, wherein to determine the deteriorating condition of the user's skin, the one or more computer programs further include instructions that, when executed by the one or more processors, cause the system to:
monitor the identified hand movement for a predefined time duration;
identify one or more changes in the identified hand movement during the predefined time duration;
correlate the identified one or more changes with the environmental conditions; and
determine the deteriorating condition of the user's skin based on the correlation.

18. The system as claimed in claim 10, wherein the one or more computer programs further include instructions that, when executed by the one or more processors, cause the system to:
generate, via at least one of the wearable device or the mobile device of the user, an alert based on the generated personalized report.

19. One or more non-transitory computer-readable storage media storing one or more computer programs including computer-executable instructions that, when executed by one or more processors of an electronic device for generating a personalized report on skin condition of a user using a wearable device connected to a mobile device of the user, cause the electronic device to perform operations, the operations comprising:
monitoring, using the wearable device, skin attributes and environmental conditions associated with the user;
determining, based on sensor-related data from the wearable device, a distance and an angle of the wearable device with reference to a position of the mobile device to identify hand movement of the user;
determining, based on the sensor-related data from the wearable device, one or more parameters associated with the identified hand movement of the user;
classifying the identified hand movement of the user as a scratching signature by correlating the determined one or more parameters with corresponding reference parameters associated with one or more predefined scratching patterns;
determining, using the wearable device, one or more scratching characteristics associated with the scratching signature of the user;
determining a level of scratching and a deteriorating condition of the user's skin by comparing the one or more scratching characteristics with one or more predefined scratching characteristics and associated skin diseases information stored in a database; and
generating the personalized report on the skin condition based on at least one of the determined level of scratching, the deteriorating condition of the user's skin, the skin attributes, and the environmental conditions associated with the user.

20. The one or more non-transitory computer-readable storage media of claim 19, the operations further comprising generating, via at least one of the wearable device or the mobile device of the user, an alert based on the generated personalized report.
